(19) United States
(12) Patent Application Publication
(10) Pub. No.: US 2008/0051430 A1
(43) Pub. Date: Feb. 28, 2008

(54) Acylated Piperidine Derivatives as Melanocortin 4-Receptor Agonists

(76) Inventors: Khaled J. Barakat, Brooklyn, NY (US); Liangqin Guo, Edison, NJ (US); Jian Liu, Edison, NJ (US); Ravi P. Nargund, East Brunswick, NJ (US); Iyassu K. Sebhat, New York, NY (US); Zhixiong Ye, Princeton, NJ (US)

Correspondence Address: MERCK AND CO., INC, P O BOX 2000, RAHWAY, NJ, 07065-0907, US

(21) Appl. No.: 11/632,001

(22) PCT Filed: Jul. 13, 2005
(86) PCT No.: PCT/US05/24806

§ 371 Date: Jan. 9, 2007

Related U.S. Application Data
(60) Provisional application No. 60/588,494, filed on Jul. 16, 2004.

(51) Int. Cl. A61K 31/451; A61P 15/00; A61P 15/10; A61P 3/04; C07D 401/06; C07D 411/14

(52) U.S. Cl. 514/316; 514/326; 546/189; 546/208; 546/210

(57) ABSTRACT

Certain novel N-acylated piperidine derivatives are agonists of the human melanocortin receptor(s) and, in particular, are selective agonists of the human melanocortin-4 receptor (MC-4R). They are therefore useful for the treatment, control, or prevention of diseases and disorders responsive to the activation of MC-4R, such as obesity, diabetes, sexual dysfunction, including erectile dysfunction and female sexual dysfunction.

### FIELD OF THE INVENTION

The present invention relates to acylated piperidine derivatives, their synthesis, and their use as melanocortin receptor (MC-R) modulators. More particularly, the compounds of the present invention are selective agonists of the melanocortin-4 receptor (MC-4R) and are thereby useful for the treatment of disorders responsive to the activation of MC-4R, such as obesity, diabetes, male sexual dysfunction, and female sexual dysfunction.

### BACKGROUND OF THE INVENTION

Obesity is a major health concern in Western societies. It is estimated that about 97 million adults in the United States are overweight or obese. The medical problems associated with obesity, which can be serious and life-threatening, include hypertension; type 2 diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arterioscelerosis; heart disease; abnormal heart rhythms; and heart arythmias (Kopelman, P. G., Nature 404, 635-643 (2000)). Obesity is further associated with premature death and with a significant increase in mortality and morbidity from stroke, myocardial infarction, congestive heart failure, coronary heart disease, and sudden death. Obesity also exacerbates many health problems, both independently and in association with other diseases.

Pro-opiomelanocortin (POMC) derived peptides are known to affect food intake. Five distinct MC-R's have thus far been identified, and these are expressed in different tissues. MC-1R is mainly expressed in melanocytes, and has been found to affect coat color by controlling phaeomelanin to eumelanin conversion through control of tyrosinase. MC-2R is expressed in the adrenal gland and represents the ACTH receptor. MC-3R is expressed in the brain, gut, and placenta and may be involved in the control of food intake and thermogenesis. MC-4R is uniquely expressed in the brain, and its inactivation was shown to cause obesity (A. Kask, et al., “Selective antagonist for the melanocortin-4 receptor (HS014) increases food intake in free-feeding rats,” Biochem. Biophys. Res. Commun., 245: 90-93 (1998)). MC-5R is expressed in many tissues, including white fat, placenta and exocrine glands, and in the brain. MC-5R knockout mice reveal reduced sebaceous gland lipid production (Chen et al., Cell, 91: 789-798 (1997)). A specific single MC-R that may be targeted for the control of obesity has not yet been identified, although evidence has been presented that MC-4R signalling is important in mediating feed behavior (S. Q. Giraudo et al., “Feeding effects of hypothalamic injection of melanocortin-4 receptor ligands,” Brain Research, 80: 302-306 (1998)).

Weight loss drugs that are currently used to treat obesity have limited efficacy. Studies of the weight loss medications orlistat (Davidson, M. H. et al. (1999) JAMA 281:23542), dexfenfluramine (Guy Grand, B. et al. (1989) Lancet 2:1142-5), sibutramine (Bray, G. A. et al. (1999) Obes. Res. &:189-98) and phentermine (Douglas, A. et al. (1983) Int. J. Obes. 7:591-5) have demonstrated a limited weight loss of about 5%-10% of body weight for drug compared to placebo. The side effects of these anti-obesity agents further limit their use. Dexfenfluramine was withdrawn from the market because of suspected heart valvulopathy; orlistat is limited by gastrointestinal side effects; the use of topiramate is limited by central nervous system effects; and the use of sibutramine is limited by its cardiovascular side effects which have led to reports of deaths and its withdrawal from the market in Italy.

There is a need for a weight loss treatment with enhanced efficacy and fewer undesirable side effects. The instant invention addresses this problem by providing melanocortin receptor (MC-R) agonists, and in particular selective agonists of the melanocortin-4 receptor (MC-4R), useful in the treatment and prevention of obesity and obesity-related disorders, including diabetes.

Melanocortin receptor involvement in male and female sexual dysfunction has also been reported. Approximately 140 million men worldwide suffer from impotency or erectile dysfunction. Erectile dysfunction or “impotence” denotes the medical condition of inability to achieve penile erection sufficient for successful sexual intercourse. Erectile dysfunction can arise from either organic or psychogenic causes, with about 20% of such cases being purely psychogenic in origin. Erectile dysfunction increases from 40% at age 40, to 67% at age 75, with over 75% occurring in men over the age of 50.

Synthetic melanocortin receptor agonists (melanotropic peptides) have been found to initiate erections in men with psychogenic erectile dysfunction [See H. Wessells et al., “Synthetic Melanotropic Peptide Initiates Erections in Men With Psychogenic Erectile Dysfunction: Double-Blind, Placebo Controlled Crossover Study,” J. Urol., 160: 389-393 (1998); Fifteenth American Peptide Symposium, Jun. 14-19, 1997 (Nashville Tenn.)]. Activation of melanocortin receptors of the brain appears to cause normal stimulation of sexual arousal. In the above study, the centrally acting α-melanocyte-stimulating hormone analog, melanotan-II (MT-II), exhibited a 75% response rate when injected intramuscularly or subcutaneously to males with psychogenic erectile dysfunction. MT-II (also referred to as PT-14; Erectide®) is a synthetic cyclic heptapeptide, Ac-Nle-c[Asp-His-DPhe-Arg-Trp-Lys]-NH₂, which is a non-selective MC-1R, -3R, -4R, and -5R agonist (Dorr et al., Life Sciences, Vol. 58, 1777-1784, 1996). Drugs to treat erectile dysfunction act either peripherally or centrally and are also classified according to whether they “initiate” a sexual response or “facilitate” a sexual response to prior stimulation [for a discussion, see “A Therapeutic Taxonomy of Treatments for Erectile Dysfunction: An Evolutionary Imperative,” Int. J. Impotence Res., 9: 115-121 (1997)]. MT-II is considered to be an “initiator” of the sexual response. The time to onset of erection with this drug is relatively short (10-20 minutes) with a duration of action approximately 2.5 hours. Adverse reactions observed with MT-II include nausea, flushing, loss of appetite, stretching, and yawning and may be the result of activation of MC-1R, MC-2R, MC-3R, and/or MC-5R. MT-II must be administered parenterally, such as by subcutaneous, intravenous, or intramuscular route, since it is not absorbed into the systemic circulation when given by the oral route.

MT-II's erectogenic properties apparently are not limited to cases of psychogenic erectile dysfunction in that men with a variety of organic risk factors developed penile erections upon subcutaneous injection of the compound; moreover, the level of sexual desire was significantly higher after MT-II administration than after placebo [see H. Wessells, “Effect of an Alpha-Melanocyte Stimulating Hormone Analog on Penile Erection and Sexual Desire in Men with Organic Erectile Dysfunction,” Urology, 56: 641-646 (2000)].

Compositions of melanotropic peptides and methods for the treatment of psychogenic erectile dysfunction are disclosed in U.S. Pat. No. 5,576,290, assigned to Competitive Technologies. Methods of stimulating sexual response in females using melanotropic peptides have been disclosed in U.S. Pat. No. 6,051,555.

Spiropiperidine, piperidine and piperazine derivatives have been disclosed in U.S. Pat. Nos. U.S. Pat. No. 6,294,534, U.S. Pat. No. 6,350,760, U.S. Pat. No. 6,376,509, U.S. Pat. No. 6,410,548, U.S. Pat. No. 6,458,790, U.S. Pat. No. 6,472,398; in U.S. Patent Application Publication Nos. US2002/0004512, US2002/0019523, US2002/0137664, US 2003/0092732, US2003/0236262, US2003/0225060; and in International Patent Publications WO 99/64002, WO 00174679, WO 01/058891, WO 01/70708, WO 01/70337, WO 01/91752, WO 02/015909, WO 02/067869, WO 02/068387, WO 02/068388, WO 02/079146, WO 03/007949, WO 03/009847, WO 03/057671, WO 03/068738, WO 03/092690, and WO 04/024720, as agonists of the melanocortin receptor(s) and particularly as selective agonists of the MC-4R receptor and thereby useful for the treatment of diseases and disorders, such as obesity, diabetes, and sexual dysfunction, including erectile dysfunction and female sexual dysfunction.

Other pharmacological approaches to the treatment of erectile dysfunction have been described [see, e.g., “Latest Findings on the Diagnosis and Treatment of Erectile Dysfunction,” Drug News & Perspectives, 9: 572-575 (1996); “Oral Pharmacotherapy in Erectile Dysfunction,” Current Opinion in Urology, 7: 349-353 (1997)].

Because of the unresolved deficiencies of the various pharmacological agents discussed above, there is a continuing need in the medical arts for improved methods and compositions to treat individuals suffering from psychogenic and/or organic sexual dysfunction. Such methods should have wider applicability, enhanced convenience and ease of compliance, short onset of action, reasonably long duration of action, and minimal side effects with few contraindications, as compared to agents now available. The instant invention addresses this problem by providing melanocortin receptor (MC-R) agonists, and in particular selective agonists of the melanocortin-4 receptor (MC-4R), useful in the treatment and prevention of sexual dysfunction, including male erectile dysfunction and female sexual dysfunction.

It is therefore an object of the present invention to provide acylated piperidine derivatives which are melanocortin receptor agonists and thereby useful to treat obesity, diabetes, male sexual dysfunction, and female sexual dysfunction.

It is another object of the present invention to provide acylated piperidine derivatives which are selective agonists of the melanocortin-4 (MC-4R) receptor.

It is another object of the present invention to provide pharmaceutical compositions comprising the melanocortin receptor agonists of the present invention with a pharmaceutically acceptable carrier.

It is another object of the present invention to provide methods for the treatment or prevention of disorders, diseases, or conditions responsive to the activation of the melanocortin-4 receptor in a mammal in need thereof by administering the compounds and pharmaceutical compositions of the present invention.

It is another object of the present invention to provide methods for the treatment or prevention of obesity, diabetes mellitus, male sexual dysfunction, and female sexual dysfunction by administering the compounds and pharmaceutical compositions of the present invention to a mammal in need thereof.

It is another object of the present invention to provide methods for the treatment of erectile dysfunction by administering the compounds and pharmaceutical compositions of the present invention to a mammal in need thereof.

These and other objects will become readily apparent from the detailed description that follows.

### SUMMARY OF THE INVENTION

The present invention relates to novel 4-phenyl substituted piperidines of structural formula I:

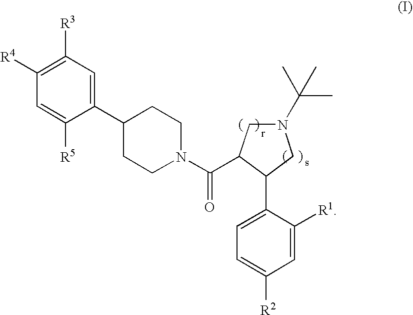

These piperidine derivatives are effective as melanocortin receptor agonists and are particularly effective as selective melanocortin-4 receptor (MC-4R) agonists. They are therefore useful for the treatment and/or prevention of disorders responsive to the activation of MC-4R, such as obesity, diabetes as well as male and female sexual dysfunction, in particular, male erectile dysfunction.

The present invention further relates to pharmaceutical compositions comprising the compounds of the present invention and a pharmaceutically acceptable carrier.

The present invention also relates to methods for the treatment or prevention of disorders, diseases, or conditions responsive to the activation of the melanocortin-4 receptor in a mammal in need thereof by administering the compounds and pharmaceutical compositions of the present invention.

The present invention also relates to methods for the treatment or prevention of obesity, diabetes mellitus, male sexual dysfunction, and female sexual dysfunction by administering the compounds and pharmaceutical compositions of the present invention.

The present invention also relates to methods for treating erectile dysfunction by administering the compounds and pharmaceutical compositions of the present invention.

The present invention also relates to methods for treating erectile dysfunction by administering the compounds of the present invention in combination with a therapeutically effective amount of another agent known to be useful to treat the condition.

The present invention also relates to methods for treating or preventing obesity by administering the compounds of the present invention in combination with a therapeutically effective amount of another agent known to be useful to prevent or treat the condition.

The present invention also relates to methods for treating or preventing diabetes by administering the compounds of the present invention in combination with a therapeutically effective amount of another agent known to be useful to prevent or treat the condition.

### DETAILED DESCRIPTION OF THE INVENTION

The present invention relates to 4-substituted N-acylated piperidine derivatives useful as melanocortin receptor agonists, in particular, as selective MC-4R agonists. Compounds of the present invention are described by structural formula I: 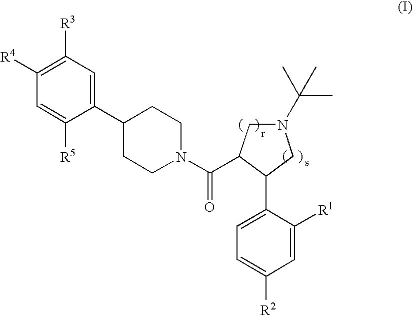
or a pharmaceutically acceptable salt thereof; wherein
R¹ and R² are selected from the group consisting of:

(1) halogen,

(2) CF₃,

(3) CH₃, and

(4) OCH₃;
R³ and R⁴ are independently selected from the group consisting of:

(1) —C₁₋₄alkyl,

(2) —CF₃,

(3) halogen,

(4) —OC₁₋₄ alkyl,

(5) —OCF₃,

(6) —OCHF₂,

(7) —S(O)ₚC₁₋₄alkyl, and

(8) —CN,

wherein alkyl is unsubstituted or substituted with one to three substituents independently selected from halogen, hydroxy, oxo, C₁₋₄ alkyl, trifluoromethyl, and C₁₋₄ alkoxy, or wherein the R³ and R⁴ substituents taken together with the carbons to which they are attached form a 4-6 membered ring optionally containing a heteroatom selected from O, S, —NH, and —NC₁₋₄alkyl;
R⁵ is selected from the group consisting of:

(1) —C₁₋₈ alkyl,

(2) —(CH₂)ₙ-heteroaryl,

(3) —(CH₂)ₙheterocycloalkyl,

(4) halogen,

(5) —OR⁶,

(6) —(CH₂)ₙC(O)R⁶,

(7) —(CH₂)ₙOC(O)R⁶,

(8) —(CH₂)ₙC(O)OR⁶,

(9) —(CH₂)ₙC≡N,

(10) —(CH₂)ₙN(R⁶)₂,

(11) —(CH₂)ₙC(O)N(R⁶)₂,

(12) —(CH₂)ₙNR⁶C(O)R⁶,

(13) —(CH₂)ₙNR⁶C(O)OR⁶,

(14) —(CH₂)ₙNR⁶C(O)-heteroaryl,

(15) —(CH₂)ₙNR⁶C(O)N(R⁶)₂,

(16) —(CH₂)ₙNR⁶-heteroaryl,

(17) —(CH₂)ₙC(O)NR⁶N(R⁶)₂,

(18) —(CH₂)ₙC(O)NR⁶NR⁶C(O)R⁶,

(19) —(CH₂)ₙNR⁶S(O)ₚR⁶,

(20) —(CH₂)ₙS(O)ₚN(R⁶)₂,

(21) —(CH₂)ₙS(O)ₚR⁶,

(22) —O(CH₂)ₙC(O)N(R⁶)₂,

(23) —(CH₂)ₙCF₃, and

(24) —O(CH₂)ₙCF_{3,}

wherein heteroaryl is unsubstituted or substituted with one to three substituents independently selected from halogen, hydroxy, C₁₋₄ alkyl, trifluoromethyl, and C₁₋₄ alkoxy, and wherein any alkyl, heterocycloalkyl, and methylene (CH₂) carbon atom in R⁵ is unsubstituted or substituted with one to two substituents independently selected from halogen, hydroxy, oxo, C₁₋₄ alkyl, trifluoromethyl, and C₁₋₄ alkoxy, or two substituents on the same R⁵ carbon atom are taken together with the carbon atom to form a 3- to 6-membered ring;
each R⁶ is independently selected from the group consisting of

(1) hydrogen,

(2) C₁₋₈ alkyl,

(3) phenyl,

(4) heteroaryl,

(5) —(CH₂)ₙheterocycloalkyl, and

(6) C₃₋₆ cycloalkyl,

wherein alkyl, phenyl, heteroaryl, heterocycloalkyl, and cycloalkyl are unsubstituted or substituted with one to three substituents independently selected from halogen, C₁₋₄ alkyl, hydroxy, and C₁₋₄ alkoxy, or two R⁶ substituents together with the atoms to which they are attached form a 4- to 8-membered mono- or bicyclic ring system optionally containing an additional heteroatom selected from O, S, —NH, and —NC₁₋₄ alkyl;
r is 1 or 2;
s is 0, 1, or 2;
n is 0, 1, 2, 3, or 4; and
p is 0, 1, or 2.

In yet a further embodiment of the compounds of the present invention, there are provided compounds of structural formula IIa or IIb of the indicated relative stereochemical configurations having the trans orientation of the difluorophenyl and piperidinecarbonyl substituents: 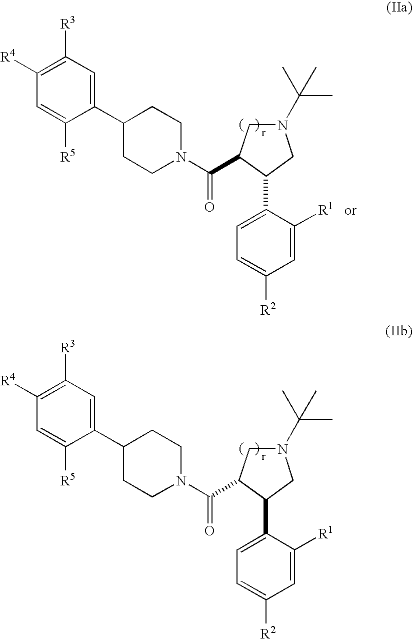
or a pharmaceutically acceptable salt thereof; wherein
R¹ and R² is selected from the group consisting of:

(1) chloro,

(2) fluoro,

(3) bromo,

(4) CF₃,

(5) CH₃, and

(6) OCH₃;
R³ and R⁴ are independently selected from the group consisting of:

(1) —C₁₋₄ alkyl,

(2) —CF₃,

(3) halogen,

(4) —OC₁₋₄ alkyl,

(5) —OCF₃,

(6) —OCHF₂,

(7) —S(O)ₚC₁₋₄ alkyl, and

(8) —CN,

wherein alkyl is unsubstituted or substituted with one to three substituents independently selected from halogen, hydroxy, OXO, C₁₋₄ alkyl, trifluoromethyl, and C₁₋₄ alkoxy, or wherein the R³ and R⁴ substituents taken together with the carbons to which they are attached form a 4-6 membered ring optionally containing a heteroatom selected from O, S, —NH, and —NC₁₋₄alkyl;
R⁵ is selected from the group consisting of:

(1) —C₁₋₈ alkyl,

(2) —(CH₂)ₙ-heteroaryl,

(3) —(CH₂)ₙheterocycloalkyl,

(4) halogen,

(5) —OR⁶,

(6) —(CH₂)ₙC(O)R⁶,

(7) —(CH₂)ₙOC(O)R⁶,

(8) —(CH₂)ₙC(O)OR⁶,

(9) —(CH₂)ₙC≡N,

(10) —(CH₂)ₙN(R⁶)₂,

(11) —(CH₂)ₙC(O)N(R⁶)₂,

(12) —(CH₂)ₙNR⁶C(O)R⁶,

(13) —(CH₂)ₙNR⁶C(O)OR⁶,

(14) —(CH₂)ₙNR⁶C(O)-heteroaryl,

(15) —(CH₂)ₙNR⁶C(O)N(R⁶)₂,

(16) —(CH₂)ₙNR⁶-heteroaryl,

(17) —(CH₂)ₙC(O)NR⁶N(R⁶)₂,

(18) —(CH₂)ₙC(O)NR⁶NR⁶C(O)R⁶,

(19) —(CH₂)ₙNR⁶S(O)ₚR⁶,

(20) —(CH₂)ₙS(O)ₚN(R⁶)₂,

(21) —(CH₂)ₙS(O)ₚR⁶,

(22) —O(CH₂)ₙC(O)N(R⁶)₂,

(23) —(CH₂)ₙCF₃, and

(24) —O(CH₂)ₙCF₃,

wherein heteroaryl is unsubstituted or substituted with one to three substituents independently selected from halogen, hydroxy, C₁₋₄ alkyl, trifluoromethyl, and C₁₋₄ alkoxy, and wherein any alkyl, heterocycloalkyl, and methylene (CH₂) carbon atom in R⁵ is unsubstituted or substituted with one to two substituents independently selected from halogen, hydroxy, oxo, C₁₋₄ alkyl, trifluoromethyl, and C₁₋₄ alkoxy, or two substituents on the same R⁵ carbon atom are taken together with the carbon atom to form a 3- to 6-membered ring;
each R⁶ is independently selected from the group consisting of

(1) hydrogen,

(2) C₁₋₈ alkyl,

(3) phenyl,

(4) heteroaryl,

(5) —(CH₂)ₙheterocycloalkyl, and

(6) C₃₋₆ cycloalkyl,

wherein alkyl, phenyl, heteroaryl, heterocycloalkyl, and cycloalkyl are unsubstituted or substituted with one to three substituents independently selected from halogen, C₁₋₄ alkyl, hydroxy, and C₁₋₄ alkoxy, or two R⁶ substituents together with the atoms to which they are attached form a 4- to 8-membered mono- or bicyclic ring system optionally containing an additional heteroatom selected from O, S, —NH, and —NC₁₋₄ alkyl; and
r is 1 or 2;
n is 0, 1, 2, 3, or 4; and
p is 0, 1, or 2.

In yet a further embodiment of the compounds of the present invention, there are provided compounds of structural formula IIIa or IIIb 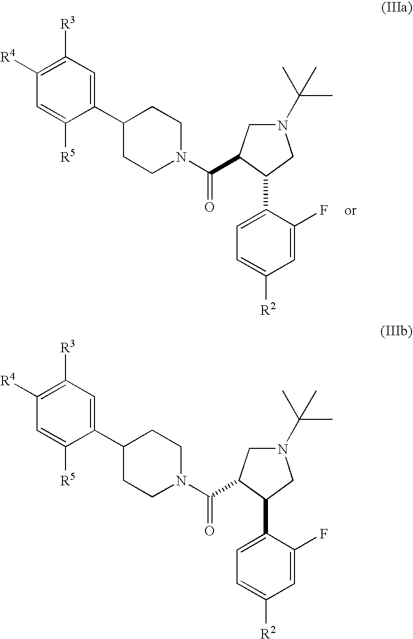
wherein R² is selected from chloro and fluoro, and R³, R⁴, and R⁵ are as defined above; and pharmaceutically acceptable salts thereof.

In yet a further embodiment of the compounds of the present invention, there are provided compounds of structural formula IVa or IVb 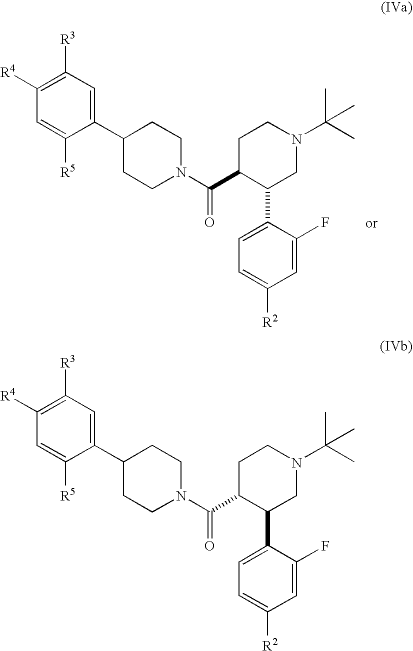
wherein R² is selected from chloro and fluoro, and R³, R⁴, and R⁵ are as defined above; and pharmaceutically acceptable salts thereof.

In a class of the embodiments of the present invention, R¹ is selected from the group consisting of: methyl, chloro, and fluoro. In a subclass of this class, R¹ is fluoro.

In another class of the embodiments of the present invention, R² is selected from the group consisting of: fluoro and chloro. In a subclass of this class, R² is fluoro. In another subclass of this class, R² is chloro.

In another class of the embodiments of the present invention, R³ and R⁴ are independently selected from the group consisting of: —C₁₋₄ alkyl, —CF₃, halogen, —OC₁₋₄ alkyl, —OCF₃, —OCHF₂, —S(O)ₚC₁₋₄alkyl, and —CN, wherein alkyl is unsubstituted or substituted with one to three substituents independently selected from halogen, hydroxy, oxo, C₁₋₄ alkyl, trifluoromethyl, and C₁₋₄ alkoxy, or wherein the R³ and R⁴ substituents taken together with the carbons to which they are attached form a 4-6 membered ring optionally containing a heteroatom selected from O, S, —NH, and —NC₁₋₄alkyl. In subclass of this class, R³ and R⁴ are independently selected from the group consisting of: —C₁₋₄alkyl and halogen, wherein alkyl is unsubstituted or substituted with one to three substituents independently selected from halogen, hydroxy, oxo, C₁₋₄ alkyl, trifluoromethyl, and C₁₋₄ alkoxy, or wherein the R³ and R⁴ substituents taken together with the carbons to which they are attached form a 4-6 membered ring. In another subclass of this class, R³ and R⁴ are independently selected from the group consisting of methyl, chloro, and fluoro. In another subclass of this class, at least one of R³ and R⁴ is methyl. In another subclass of this class, R³ is chloro, and R⁴ is methyl. In another subclass of this class, R³ is methyl, and R⁴ is chloro. In another subclass of this class, R³ is fluoro, and R⁴ is methyl. In another subclass of this class, R³ is methyl, and R⁴ is fluoro. In yet another subclass of this class, both R³ and R⁴ are methyl, and the R³ and R⁴ substituents taken together with the carbons to which they are attached form a 4-6 membered ring.

In another class of the embodiments of the present invention, R⁵ is selected from the group consisting of: —C₁₋₈ alkyl, —(CH₂)ₙ-heteroaryl, —(CH₂)ₙheterocycloalkyl, halogen, —OR⁶, —(CH₂)ₙC(O)R⁶, —(CH₂)ₙOC(O)R⁶, —(CH₂)ₙC(O)OR⁶, —(CH₂)ₙC≡N, —(CH₂)ₙN(R⁶)₂, —(CH₂)ₙC(O)N(R⁶)₂, —(CH₂)ₙNR⁶C(O)R⁶, —(CH₂)ₙNR⁶C(O)OR⁶, —(CH₂)ₙNR⁶C(O)-heteroaryl, —(CH₂)ₙNR⁶C(O)N(R⁶)₂, —(CH₂)ₙNR⁶-heteroaryl, —(CH₂)ₙC(O)NR⁶N(R⁶)₂, —(CH₂)ₙC(O)NR⁶NR⁶C(O)R⁶, —(CH₂)ₙNR⁶S(O)ₚR⁶, —(CH₂)ₙS(O)ₚN(R⁶)₂, —(CH₂)ₙS(O)ₚR⁶, —O(CH₂)ₙC(O)N(R⁶)₂, —CF₃, —CH₂CF₃, —OCF₃, and —OCH₂CF₃, wherein heteroaryl is unsubstituted or substituted with one to three substituents independently selected from halogen, hydroxy, C₁₋₄ alkyl, trifluoromethyl, and C₁₋₄ alkoxy, and wherein any alkyl, heterocycloalkyl, and methylene (CH₂) carbon atom in R⁵ is unsubstituted or substituted with one to two substituents independently selected from halogen, hydroxy, oxo, C₁₋₄ alkyl, trifluoromethyl, and C₁₋₄ alkoxy. In a subclass of this class, R⁵ is selected from the group consisting of: —(CH₂)ₙ-heteroaryl, and —(CH₂)ₙNR⁶C(O)R⁶, wherein heteroaryl is unsubstituted or substituted with one to three substituents independently selected from halogen, hydroxy, C₁₋₄ alkyl, trifluoromethyl, and C₁₋₄ alkoxy, and wherein any methylene (CH₂) carbon atom in R⁵ is unsubstituted or substituted with one to two substituents independently selected from halogen, hydroxy, oxo, C₁₋₄ alkyl, trifluoromethyl, and C₁₋₄ alkoxy, or two substituents on the same R⁵ carbon atom are taken together with the carbon atom to form a 3- to 6-membered ring. In a class of this embodiment, R⁵ is selected from the group consisting of: —(CH₂)₁-heteroaryl, and —(CH₂)₁NR⁶C(O)R⁶, wherein heteroaryl is unsubstituted or substituted with one to three substituents independently selected from methyl and ethyl, and wherein any methylene (CH₂) carbon atom in R⁵ is unsubstituted or substituted with one to two substituents independently selected from methyl and ethyl. In a subclass of these classes of R⁵, any methylene (CH₂) carbon atom in R⁵ is substituted with one to two substituents independently selected from methyl and ethyl. In another subclass of these classes of R⁵, the heteroaryl is selected from the group consisting of triazole, and tetrazole.

In another class of the embodiments of the present invention, r is 1. In a subclass of this class, r is 1 and s is 1.

In another class of the embodiments of the present invention, r is 2. In a subclass of this class, r is2and s is 1.

Illustrative, but nonlimiting, examples of compounds of the present invention that are useful as melanocortin-4 receptor agonists are the following: 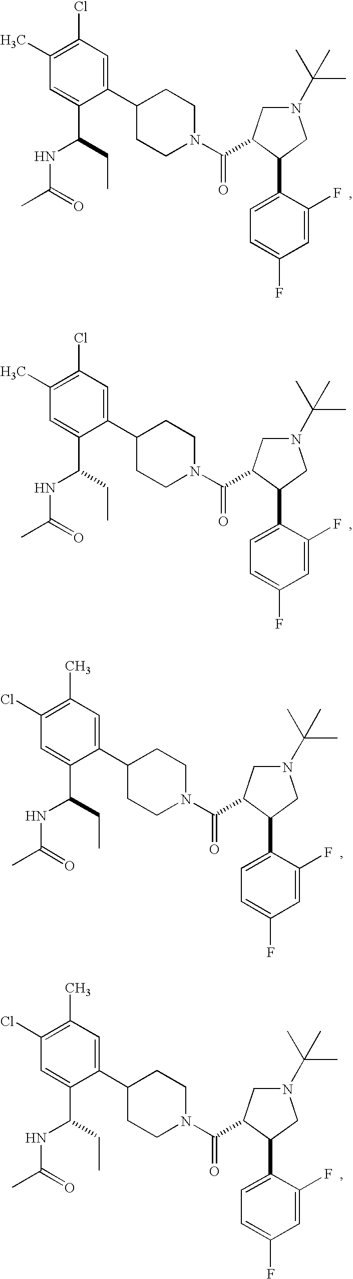 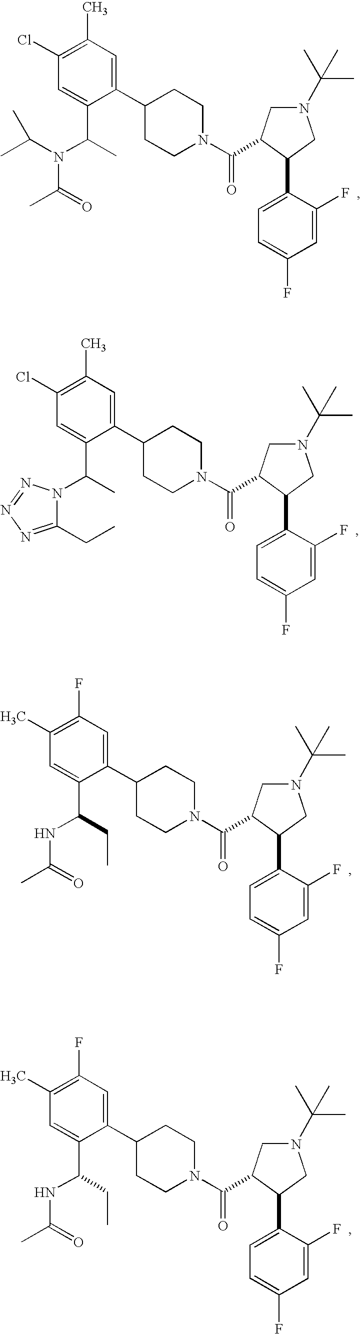 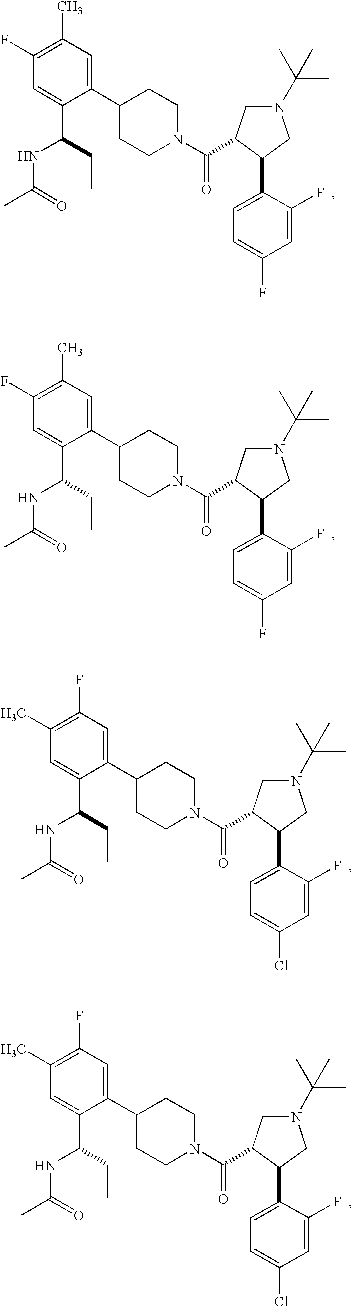 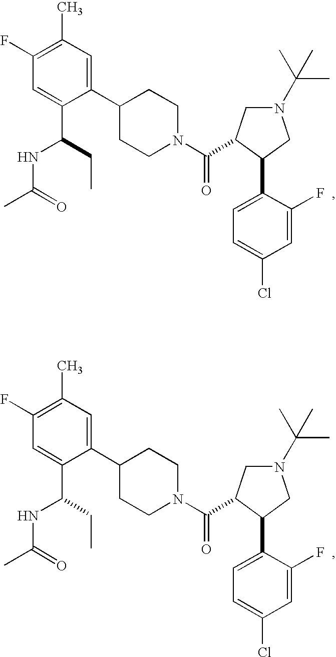
and pharmaceutically acceptable salts thereof.

Additional illustrative, but nonlimiting, examples of compounds of the present invention that are useful as melanocortin-4 receptor agonists are the following: 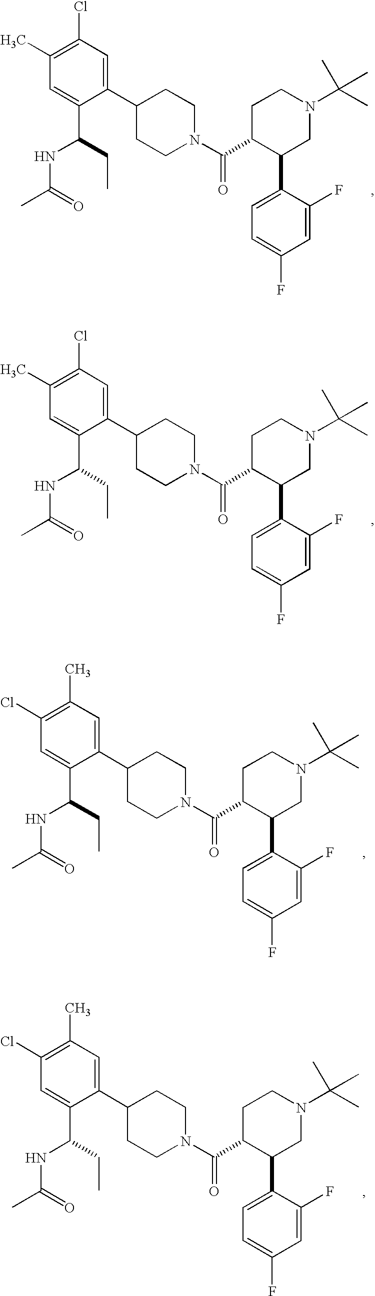 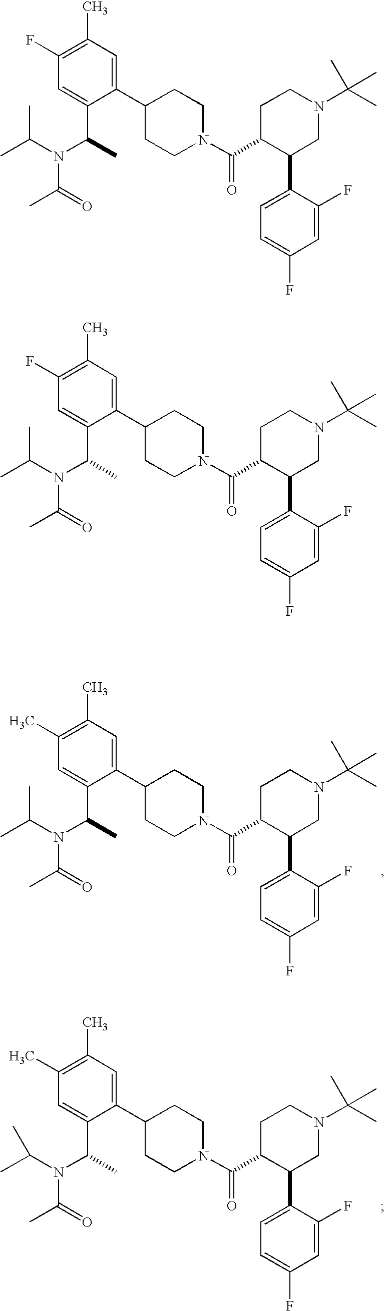
and pharmaceutically acceptable salts thereof.

The compounds of structural formula I are effective as melanocortin receptor ligands and are particularly effective as selective agonists of MC-4R. They are therefore useful for the treatment and/or prevention of disorders responsive to the activation of MC-4R, such as obesity, diabetes as well as male and/or female sexual dysfunction, in particular, erectile dysfunction, and further in particular, male erectile dysfunction.

One apsect of the present invention provides a method for the treatment or prevention of disorders, diseases or conditions responsive to the activation of the melanocortin-4 receptor in a mammal in need thereof which comprises administering to the mammal a therapeutically or prophylactically effective amount of a compound of structural formula I.

Another aspect of the present invention provides a method for the treatment or prevention of obesity in a mammal in need thereof which comprises administering to said mammal a therapeutically or prophylactically effective amount of a compound of structural formula I. Another aspect of the present invention provides a method for the treatment or prevention of diabetes in a mammal in need thereof which comprises administering to said mammal a therapeutically or prophylactically effective amount of a compound of structural formula I.

Yet another aspect of the present invention provides a method for the treatment or prevention of obesity which comprises administering to a mammal in need of such treatment or prevention a therapeutically or prophylactically effective amount of a compound of structural formula I in combination with a therapeutically effective amount of another agent known to be useful for the treatment of this condition. Another aspect of the present invention provides a method of treating or preventing diabetes or obesity in a mammal in need thereof comprising administering to the mammal a therapeutically effective amount of a compound of structural formula I in combination with an insulin sensitizer, an insulin mimetic, a sulfonylurea, an α-glucosidase inhibitor, a HMG-CoA reductase inhibitor, a serotonergic agent, a β3-adrenoreceptor agonist, a neuropeptide Y1 antagonist, a neuropeptide Y5 antagonist, a pancreatic lipase inhibitor, a cannabinoid CB₁ receptor antagonist or any alkyl, heterocycloalkyl, and methylene (CH₂) carbon atom in R⁵ is unsubstituted or inverse agonist, a melanin-concentrating hormone receptor antagonist, a bombesin receptor subtype 3 agonist, a ghrelin receptor antagonist, or a dipeptidyl peptidase IV inhibitor. Another aspect of the present invention provides a method of treating or preventing an obesity-related disorder selected from the group consisting of overeating, binge eating, and bulimia, hypertension, diabetes, elevated plasma insulin concentrations, insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, metabolic syndrome, insulin resistance syndrome, sexual and reproductive dysfunction, infertility, hypogonadism, hirsutism, obesity-related gastro-esophageal reflux, Pickwickian syndrome, cardiovascular disorders, inflammation, systemic inflammation of the vasculature, arteriosclerosis, hypercholesterolemia, hyperuricaemia, lower back pain, gallbladder disease, gout, and kidney cancer, cardiac hypertrophy and left ventricular hypertrophy, in a mammal in need thereof which comprises administering to the mammal a therapeutically or prophylactically effective amount of a compound of structural formula I.

Yet another aspect of the present invention provides a pharmaceutical composition of a compound of structural formula I further comprising a second active ingredient selected from the group consisting of an insulin sensitizer, an insulin mimetic, a sulfonylurea, an α-glucosidase inhibitor, a HMG-CoA reductase inhibitor, a serotonergic agent, a β3-adrenoreceptor agonist, a neuropeptide Y1 antagonist, a neuropeptide Y5 antagonist, a pancreatic lipase inhibitor, a cannabinoid CB₁ receptor antagonist or inverse agonist, a melanin-concentrating hormone receptor antagonist, a bombesin receptor subtype 3 agonist, a ghrelin receptor antagonist, and a dipeptidyl peptidase IV inhibitor.

Another aspect of the present invention provides a method for the treatment or prevention of male or female sexual dysfunction in a mammal in need thereof comprising administering to the mammal a therapeutically or prophylactically effective amount of a compound of structural formula I. Another aspect of the present invention provides a method for the treatment or prevention of erectile dysfunction in a mammal in need thereof comprising administering to the mammal a therapeutically or prophylactically effective amount of a compound of structural formula I.

Another aspect of the present invention provides a method for the treatment or prevention of male or female sexual dysfunction including erectile dysfunction which comprises administering to a mammal in need of such treatment or prevention a therapeutically or prophylactically effective amount of a compound of structural formula I in combination with a therapeutically effective amount of another agent known to be useful for the treatment of these conditions. Another aspect of the present invention provides a method of treating erectile dysfunction in a mammal in need thereof comprising administering to the mammal a therapeutically effective amount of a compound structural formula I in combination with a type V cyclic-GMP-selective phosphodiesterase inhibitor, an α₂-adrenergic receptor antagonist, or a dopaminergic agent. Yet another aspect of the present invention provides a pharmaceutical composition of a compound structural formula I further comprising a second active ingredient selected from the group consisting of a type V cyclic-GMP-selective phosphodiesterase inhibitor, an α₂-adrenergic receptor antagonist, and a dopaminergic agent.

Yet another aspect of the present invention provides a pharmaceutical composition comprising a compound of structural formula I and a pharmaceutically acceptable carrier.

Yet another aspect of the present invention relates to the use of a compound of formula I for the manufacture of a medicament useful for the treatment or prevention, or suppression of a disease mediated by the melanocortin-4 receptor in a mammal in need thereof. Yet another aspect of the present invention relates to the use of a compound of formula I for the manufacture of a medicament useful for the treatment or prevention, or suppression of obesity in a mammal in need thereof. Another aspect of the invention relates to the use of a compound of formula I for the manufacture of a medicament useful for the treatment or prevention of an obesity-related disorder selected from the group consisting of overeating, binge eating, and bulimia, hypertension, diabetes, elevated plasma insulin concentrations, insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, metabolic syndrome, insulin resistance syndrome, sexual and reproductive dysfunction, infertility, hypogonadism, hirsutism, obesity-related gastro-esophageal reflux, Pickwickian syndrome, cardiovascular disorders, inflammation, systemic inflammation of the vasculature, arteriosclerosis, hypercholesterolemia, hyperuricaemia, lower back pain, gallbladder disease, gout, and kidney cancer, cardiac hypertrophy and left ventricular hypertrophy. Yet another aspect of the present invention relates to the use of a compound of formula I for the manufacture of a medicament useful for the treatment or prevention, or suppression of diabetes in a mammal in need thereof. Yet another aspect of the present invention relates to the use of a compound of formula I for the manufacture of a medicament useful for the treatment or prevention, or suppression of male sexual dysfunction and female sexual dysfunction in a mammal in need thereof. Yet another aspect of the present invention relates to the use of a compound of formula I for the manufacture of a medicament useful for the treatment or prevention, or suppression of male erectile dysfunction in a mammal in need thereof.

Melanocortin receptor agonist compounds can be provided in a kit. Such a kit typically contains an active compound in dosage forms for administration. A dosage form contains a sufficient amount of active compound such that a beneficial effect can be obtained when administered to a subject during regular intervals, such as 1 to 6 times a day, during the course of 1 or more days. Preferably, a kit contains instructions indicating the use of the dosage form for weight reduction (e.g., to treat obesity or overweight) or sexual dysfunction, and the amount of dosage form to be taken over a specified time period.

Throughout the instant application, the following terms have the indicated meanings:

The term “alkyl”, as well as other groups having the prefix “alk”, such as alkoxy, alkanoyl, means carbon chains of the designated length which may be in a straight or branched configuration, or combinations thereof. Examples of alkyl groups include methyl, ethyl, n-propyl, isopropyl, n-butyl, 1-methylpropyl, 2-methylpropyl, tert-butyl, n-pentyl, 1-methylbutyl, 2-methylbutyl, 3-methylbutyl, 1,2-dimethylpropyl, 1,1-dimethylpropyl, 2,2-dimethylpropyl, n-hexyl, 1-methylpentyl, 2-methylpentyl, 3-methylpentyl, 4-methylpentyl, 1-ethylbutyl, 2-ethylbutyl, 3-ethylbutyl, 1,1-dimethyl butyl, 1,2-dimethylbutyl, 1,3-dimethylbutyl, 2,2-dimethylbutyl, 2,3-dimethylbutyl, 3,3-dimethyl butyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, 4-ethylpentyl, 1-propylbutyl, 2-propylbutyl, 3-propylbutyl, 1,1-dimethylpentyl, 1,2-dimethylpentyl, 1,3-dimethylpentyl, 1,4-dimethylpentyl, 2,2-dimethylpentyl, 2,3-dimethylpentyl. 2,4-dimethylpentyl, 3,3-dimethylpentyl, 3,4-dimethylpentyl, 4,4-dimethylpentyl, 1-methyl-1-ethylbutyl, 1-methyl-2-ethylbutyl, 2-methyl-2-ethylbutyl, 1-ethyl-2-methylbutyl, 1-ethyl-3-methylbutyl, 1,1-diethylpropyl, n-octyl, n-nonyl, and the like.

The term “alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

The term “alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

The term “halogen” includes fluorine, chlorine, bromine and iodine.

The term “C₁₋₄ alkyliminoyl” means C₁₃C(═NH)—.

The term “aryl” includes mono- or bicyclic aromatic rings containing only carbon atoms. Examples of aryl include phenyl and naphthyl.

The term “heteroaryl” includes mono- and bicyclic aromatic rings containing from 1 to 4 heteroatoms selected from nitrogen, oxygen and sulfur. Examples thereof include, but are not limited to, pyridinyl, furyl, furanyl, thienyl, pyrrolyl, oxazolyl, thiazolyl, triazolyl, triazinyl, tetrazolyl, thiadiazolyl, imidazolyl, isoxazolyl, isothiazolyl, oxadiazolyl, pyrazolyl, pyrimidinyl, pyrazinyl, pyridazinyl, quinolyl, isoquinolyl, benzimidazolyl, benzofuryl, benzothienyl, indolyl, benzthiazolyl, benzoxazolyl, and the like. In one embodiment of the present invention, heteroaryl is selected from the group consisting of pyridinyl, furyl, furanyl, thienyl, pyrrolyl, oxazolyl, thiazolyl, triazolyl, triazinyl, tetrazolyl, thiadiazolyl, imidazolyl, pyrazolyl, isoxazolyl, isothiazolyl, oxathiazolyl, pyrimidinyl, pyrazinyl, pyridazinyl, quinolyl, isoquinolyl, benzimidazolyl, benzofuryl, benzothienyl, indolyl, benzthiazolyl, and benzoxazolyl. Bicyclic heteroaromatic rings include, but are not limited to, benzothiadiazole, indole, benzothiophene, benzofuran, benzimidazole, benzisoxazole, benzothiazole, quinoline, quinazoline, benzotriazole, benzoxazole, isoquinoline, isoindoline, purine, furopyridine, thienopyridine, benzisodiazole, triazolopyrimidine, and 5,6,7,8-tetrahydroquinoline.

The term “cycloalkyl” includes mono- or bicyclic non-aromatic rings containing only carbon atoms. Examples of cycloalkyl include, but are not limited to, cyclopropyl, cyclobutyl, cyclopentyl, cyclohexyl, and cycloheptyl.

The term “heterocycloalkyl” is intended to include 3 to 10 membered mono- and bicyclic non-aromatic heterocycles containing one to four heteroatoms selected from nitrogen, oxygen and sulfur. Examples of heterocycloalkyls include, but are not limited to, azetidine, piperidine, morpholine, thiamorpholine, pyrrolidine, imidazolidine, tetrahydrofuran, piperazine, 1-thia-4-aza-cyclohexane, 1-aza-4-thia-cyclohexane, and 1,3 oxazolidine.

Certain of the above defined terms may occur more than once in the above formula and upon such occurrence each term shall be defined independently of the other; thus for example, NR⁴R⁴ may represent NH₂, NHCH₃, N(CH₃)CH₂CH₃, and the like.

The term “subject” means a mammal. One embodiment of the term “mammal” is a “human,” said human being either male or female. As such, the term “mammal” includes, but is not limited to, companion animals such as cats and dogs, as well as horses.

The term “mammal in need thereof” refers to a mammal who is in need of treatment or prophylaxis as determined by a researcher, veterinarian, medical doctor or other clinician.

The term “composition,” as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

By a melanocortin receptor “agonist” is meant an endogenous or drug substance or compound that can interact with a melanocortin receptor and initiate a pharmacological response characteristic of the melanocortin receptor. By a melanocortin receptor “antagonist” is meant a drug or a compound that opposes the melanocortin receptor-associated responses normally induced by another bioactive agent. The “agonistic” properties of the compounds of the present invention were measured in the functional assay described below. The functional assay discriminates a melanocortin receptor agonist from a melanocortin receptor antagonist.

By “binding affinity” is meant the ability of a compound/drug to bind to its biological target, in the the present instance, the ability of a compound of structural formula I to bind to a melanocortin receptor. Binding affinities for the compounds of the present invention were measured in the binding assay described below and are expressed as IC₅₀'s.

“Efficacy” describes the relative intensity with which agonists vary in the response they produce even when they occupy the same number of receptors and with the same affinity. Efficacy is the property that enables drugs to produce responses. Properties of compounds/drugs can be categorized into two groups, those which cause them to associate with the receptors (binding affinity) and those that produce a stimulus (efficacy). The term “efficacy” is used to characterize the level of maximal responses induced by agonists. Not all agonists of a receptor are capable of inducing identical levels of maximal responses. Maximal response depends on the efficiency of receptor coupling, that is, from the cascade of events, which, from the binding of the drug to the receptor, leads to the desired biological effect.

The functional activities expressed as EC₅₀'s and the “agonist efficacy” for the compounds of the present invention at a particular concentration were measured in the functional assay described below.

Optical Isomers—Diastereomers—Geometric Isomers—Tautomers

Compounds of structural formula I contain one or more asymmetric centers and can thus occur as racemates and racernic mixtures, single enantiomers, diastereomeric mixtures and individual diastereomers. The present invention is meant to comprehend all such isomeric forms of the compounds of structural formula I. For example, compound V: 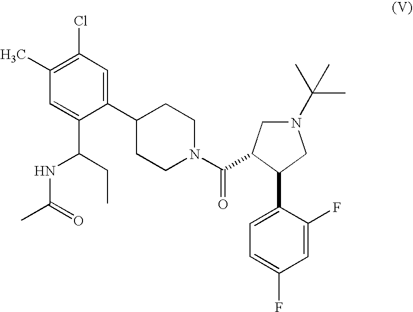
corresponds to the diastereomers Va and Vb:

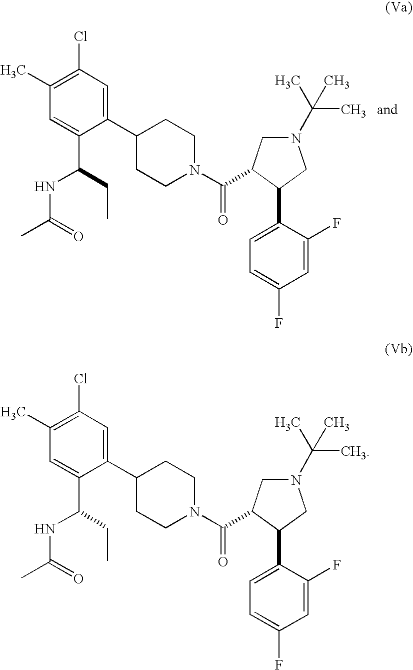

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Alternatively, any stereoisomer or diastereomer of a compound of the general formulae I, IIa, IIb, IIIa, IIIb, IVa, IVb, Va, and Vb may be obtained by stereospecific synthesis using optically pure starting materials or reagents of known absolute configuration.

Some of the compounds described herein may exist as tautomers such as keto-enol tautomers. The individual tautomers as well as mixtures thereof are encompassed within the compounds of structural formula I. Some of the compounds described herein contain olefinic double bonds, and unless specified otherwise, are meant to include both E and Z geometric isomers.

Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Particularly preferred are citric, fumaric, hydrobromic, hydrochloric, maleic, phosphoric, sulfuric, and tartaric acids.

It will be understood that, as used herein, references to the compounds of Formula I are meant to also include the pharmaceutically acceptable salts, such as the hydrochloride salts.

Utility

Compounds of formula I are melanocortin receptor agonists and as such are useful in the treatment, control or prevention of diseases, disorders or conditions responsive to the activation of one or more of the melanocortin receptors including, but are not limited to, MC-1, MC-2, MC-3, MC-4, or MC-5. Such diseases, disorders or conditions include, but are not limited to, obesity, diabetes mellitus, hypertension, hyperlipidemia, osteoarthritis, cancer, gall bladder disease, sleep apnea, depression, anxiety, compulsion, neuroses, insomnia/sleep disorder, substance abuse, pain, male and female sexual dysfunction, fever, inflammation, immunemodulation, rheumatoid arthritis, skin tanning, acne and other skin disorders, neuroprotective and cognitive and memory enhancement including the treatment of Alzheimer's disease. Some compounds encompassed by formula I show highly selective affinity for the melanocortin-4 receptor (MC-4R) relative to MC-1R, MC-2R, MC-3R, and MC-5R, which makes them especially useful in the prevention and treatment of obesity, as well as male and/or female sexual dysfunction, including erectile dysfunction.

The compositions of the present invention are useful for the treatment or prevention of disorders associated with excessive food intake, such as obesity and obesity-related disorders. The obesity herein may be due to any cause, whether genetic or environmental.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricaemia, lower back pain, gallbladder disease, gout, kidney cancer, and increased anesthetic risk. The compositions of the present invention are also useful for reducing the risk of secondary outcomes of obesity, such as reducing the risk of left ventricular hypertrophy. The compositions of the present invention are also useful to treat Alzheimer's disease.

The term “metabolic syndrome”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

Treatment of diabetes mellitus refers to the administration of a compound or combination of the present invention to treat diabetes. One outcome of treatment may be decreasing the glucose level in a subject with elevated glucose levels. Another outcome of treatment may be improving glycemic control. Another outcome of treatment may be decreasing insulin levels in a subject with elevated insulin levels. Another outcome of treatment may be decreasing plasma triglycerides in a subject with elevated plasma triglycerides. Another outcome of treatment may be lowering LDL cholesterol in a subject with high LDL cholesterol levels. Another outcome of treatment may be increasing HDL cholesterol in a subject with low HDL cholesterol levels. Another outcome may be decreasing the LDL/HDL ratio in a subject in need thereof. Another outcome of treatment may be increasing insulin sensivity. Another outcome of treatment may be enhancing glucose tolerance in a subject with glucose intolerance. Another outcome of treatment may be decreasing insulin resistance in a subject with increased insulin resistance or elevated levels of insulin. Another outcome may be decreading triglycerides in a subject with elevated triglycerides. Yet another outcome may be improving LDL cholestrol, non-HDL cholesterol, triglyceride, HDL cholesterol or other lipid analyte profiles.

Prevention of diabetes mellitus refers to the administration of a compound or combination of the present invention to prevent the onset of diabetes in a mammal at risk thereof.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus-type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

Treatment of obesity and obesity-related disorders refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in subjects in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovary disease, cardiovascular diseases, osteoarthritis, dermatological disorders, hypertension, insulin resistance, hypercholesterolemia, hypertriglyceridemia, and cholelithiasis.

“Male sexual dysfunction” includes impotence, loss of libido, and erectile dysfunction.

“Erectile dysfunction” is a disorder involving the failure of a male mammal to achieve erection, ejaculation, or both. Symptoms of erectile dysfunction include an inability to achieve or maintain an erection, ejaculatory failure, premature ejaculation, or inability to achieve an orgasm. An increase in erectile dysfunction and sexual dysfunction can have numerous underlying causes, including but not limited to (1) aging, (b) an underlying physical dysfunction, such as trauma, surgery, and peripheral vascular disease, and (3) side-effects resulting from drug treatment, depression, and other CNS disorders.

Treatment of male sexual dysfunction refers to the administration of a compound or combination of the present invention to treat impotence and/or loss of libido, and/or erectile dysfunction in a male mammal in need thereof. One outcome of treatment may be a decrease in impotence. Another outcome of treatment may be an increase in libido. Yet another outcome of treatment may be a decrease in the magnitude or frequency of erectile dysfunction.

Treatment of male sexual dysfunction refers to the administration of a compound or combination of the present invention to treat one or more of the symptoms of male sexual dysfunction in a male mammal in need thereof. One outcome of treatment may be increasing the ability to achieve an erection. Another outcome of treatment may be increasing the ability to maintain an erection. Another outcome of treatment may be reducing ejaculatory failure. Another outcome of treatment may be decreasing premature ejaculation. Yet another outcome of treatment may be increasing the ability to achieve an orgasm.

Prevention of male sexual dysfunction and male erectile dysfunction refers to the administration of the compounds or combinations of the present invention to prevent the symptoms of sexual dysfunction and erectile dysfunction in a male mammal at risk thereof.

“Female sexual dysfunction” can be seen as resulting from multiple components including dysfunction in desire, sexual arousal, sexual receptivity, and orgasm related to disturbances in the clitoris, vagina, periurethral glans, and other trigger points of sexual function. In particular, anatomic and functional modification of such trigger points may diminish the orgasmic potential in breast cancer and gynecologic cancer patients. Treatment of female sexual dysfunction with an MC-4 receptor agonist can result in improved blood flow, improved lubrication, improved sensation, facilitation of reaching orgasm, reduction in the refractory period between orgasms, and improvements in arousal and desire. In a broader sense, “female sexual dysfunction” also incorporates sexual pain, premature labor, and dysmenorrhea.

The terms “administration of” and or “administering” a compound should be understood to mean providing a compound of the invention or a prodrug of a compound of the invention to a subject in need of treatment.

The administration of the compounds of the present invention in order to practice the present methods of therapy is carried out by administering a therapeutically effective amount of the compound to a subject in need of such treatment or prophylaxis. The need for a prophylactic administration according to the methods of the present invention is determined via the use of well known risk factors.

The term “therapeutically effective amount” as used herein means the amount of the active compound that will elicit the biological or medical response in a tissue, system, subject, mammal, or human that is being sought by the researcher, veterinarian, medical doctor or other clinician, which includes alleviation of the symptoms of the disorder being treated. The novel methods of treatment of this invention are for disorders known to those skilled in the art.

The term “prophylactically effective amount” as used herein means the amount of the active compound that will elicit the biological or medical response in a tissue, system, subject, mammal, or human that is being sought by the researcher, veterinarian, medical doctor or other clinician, to prevent the onset of the disorder in subjects as risk for obesity or the disorder.

The therapeutically or prophylactically effective amount, or dosage, of an individual compound is determined, in the final analysis, by the physician in charge of the case, but depends on factors such as the exact disease to be treated, the severity of the disease and other diseases or conditions from which the subject suffers, the chosen route of administration, other drugs and treatments which the subject may concomitantly require, and other factors in the physician's judgement.

Administration and Dose Ranges

Any suitable route of administration may be employed for providing a mammal, especially a human with an effective amount, or dosage, of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of Formula I are administered orally or topically.

When treating obesity, in conjunction with diabetes and/or hyperglycemia, or alone, generally satisfactory results are obtained when the compounds of the present invention are administered at a daily dosage of from about 0.001 milligram to about 100 milligrams per kilogram of body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response. It may be necessary to use dosages outside these limits in some cases.

When treating diabetes mellitus and/or hyperglycemia, as well as other diseases or disorders, such as obesity-related disorders, for which compounds of formula I are useful, generally satisfactory results are obtained when the compounds of the present invention are administered at a daily dosage of from about 0.001 milligram to about 100 milligram per kilogram of body weight, preferably given in a single dose or in divided doses two to six times a day, or in sustained release form. In the case of a 70 kg adult human, the total daily dose will generally be from about 0.07 milligrams to about 350 milligrams. This dosage regimen may be adjusted to provide the optimal therapeutic response. It may be necessary to use dosages outside these limits in some cases.

For the treatment of sexual dysfunction compounds of the present invention are given in a dose range of 0.001 milligram to about 100 milligram per kilogram of body weight, preferably as a single dose orally or as a nasal spray.

In the case where an oral composition is employed, a suitable dosage range is, e.g. from about 0.01 mg to about 1500 mg of a compound of Formula I per day, preferably from about 0.1 mg to about 10 mg per day. For oral administration, the compositions are preferably provided in the form of tablets containing from 0.01 to 1,000 mg, preferably 0.01, 0.05, 0.1, 0.5, 1, 2.5, 5, 10, 15, 20, 25, 30, 40, 50, 100, 250, 500, 750, 1000, 1250 or 1500 milligrams of the active ingredient for the symptomatic adjustment of the dosage to the subject to be treated.

For use where a composition for intravenous administration is employed, a suitable dosage range is from about 0.001 mg to about 100 mg (preferably from 0.01 mg to about 50 mg, more preferably 0.1 mg to 10 mg) of a compound of Formula I per kg of body weight per day. This dosage regimen may be adjusted to provide the optimal therapeutic response. It may be necessary to use dosages outside these limits in some cases.

The magnitude of prophylactic or therapeutic dosage of the compounds of the present invention will, of course, vary depending on the particular compound employed, the mode of administration, the condition being treated and the severity of the condition being treated. It will also vary according to the age, weight and response of the individual subject. Such dosage may be ascertained readily by a person skilled in the art.

Combination Therapy

Compounds of structural formula I may be used in combination with other drugs that are used in the treatment/prevention/suppression or amelioration of the diseases or conditions for which compounds of structural formula I are useful. Such other drugs may be administered, by a route and in an amount commonly used therefor, contemporaneously or sequentially with a compound of structural formula I. When a compound of structural formula I is used contemporaneously with one or more other drugs, a pharmaceutical composition containing such other drugs in addition to the compound of structural formula I is preferred. When a composition of the present invention is used contemporaneously with one or more other drugs, a pharmaceutical composition in unit dosage form containing such other drugs and the composition of the present invention is preferred. However, the combination therapy also includes therapies in which the composition of the present invention and one or more other drugs are administered on different overlapping schedules. It is also contemplated that when used in combination with one or more other active ingredients, the composition of the present invention and the other active ingredients may be used in lower doses than when each is used singly. Accordingly, the pharmaceutical compositions of the present invention include those that also contain one or more other active ingredients, in addition to a compound of structural formula I.

Examples of other active ingredients that may be combined with a compound of structural formula I for the treatment or prevention of obesity and/or diabetes, either administered separately or in the same pharmaceutical compositions, include, but are not limited to:

(a) insulin sensitizers including (i) PPARγ agonists such as the glitazones (e.g. ciglitazone; darglitazone; troglitazone, pioglitazone, englitazone, isaglitazone (MCC-555), BRL49653, rosiglitazone; CLX-0921; 5-BTZD), GW-0207, LG-100641, and LY-300512, and the like), and compounds disclosed in WO97/10813, WO97/27857, 97/28115, 97/28137 and 97/27847;
ii) biguanides such as metformin (Glucophage®), buformin, and phenformin;

(b) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH₂);

(c) sulfonylureas, such as tolbutamide and glipizide, acetohexamide; chlorpropamide; diabinese; glibenclamide; glyburide; glimepiride; gliclazide; glipentide; gliquidone; glisolamide; and tolazamide;

(d) α-glucosidase inhibitors (such as acarbose, adiposine; camiglibose; emiglitate; miglitol; voglibose; pradimicin-Q; salbostatin; CKD-71 1; MDL-25,637; MDL-73,945; and MOR 14, and the like);

(e) cholesterol lowering agents such as (i) HMG-CoA reductase inhibitors (lovastatin, simvastatin, pravastatin, fluvastatin, atorvastatin, itavastatin, rivastatin, rosuvastatin, ZD-4522, and other statins), (ii) sequestrants (cholestyramine, colestipol and a dialkylaminoalkyl derivatives of a cross-4inked dextran, colesevelum, Colestid®; LoCholest®, and the like, (ii) nicotinyl alcohol nicotinic acid or a salt thereof, (iii) proliferator-activater receptor α agonists such as fenofibric acid derivatives (gemfibrozil, clofibrate, fenofibrate and benzafibrate), (iv) inhibitors of cholesterol absorption for example beta-sitosterol, stanol esters, sterol glycosides such as tiqueside; and azetidinones such as ezetirnibe, efucirnibe, KY 505, SMP797, and the like, and (acyl CoA:cholesterol acyltransferase) inhibitors for example melinamide, and avasimibe, (v) anti-oxidants such as probucol, (vi) vitamin E, and (vii) thyromimetics;

(f) PPARδ agonists, such as those disclosed in WO97/28149, and such as GW 501516, and GW 590735, and the like;

(g) anti-obesity serotonergic agents, such as fenfluramine, dexfenfluramine, phentermine, and sibutramine;

(h) β3-adrenoreceptor agonists, such as AD9677/TAK677 (Dainippon/Takeda), CL-316,243, SB 418790, BRL-37344, L-796568, BMS-196085, BRL-35135A, CGP12177A, GW 427353, BTA-243, Trecadrine, Zeneca D7114, SR 59119A, and such as those disclosed in U.S. patent application Ser. No. 5,705,515, and U.S. Pat. No. 5,451,677 and PCT Patent Publications WO94/18161, WO95/29159, WO97/46556, WO98/04526 and WO98/32753, WO 01/74782, and WO 02/32897;

(i) pancreatic lipase inhibitors, such as orlistat (Xenical®), Triton WR1339, RHC80267, lipstatin, tetrahydrolipstatin, teasaponin, diethyl-umbelliferyl phosphate, FL-386, WAY-121898, Bay-N-3176, valilactone, esteracin, ebelactone A, ebelactone B, and RHC 80267,and those disclosed in PCT Application No. WO 01177094, and U.S. Pat. Nos. 4,598,089. 4,452,813, 5,512,565, 5,391,571, 5,602,151, 4,405,644, 4,189,438, and 4,242,453;

(j) feeding behavior modifying agents, such as neuropeptideY Y1 and Y5 antagonists, such as those disclosed in WO 97/19682, WO 97/20820, WO 97/20821, WO 97/20822, WO 97/20823, WO 01/14376, and U.S. Pat. No. 6,191,160; neuropeptide Y1 antagonists, such as BIBP3226, J-1 15814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528; and neuropeptide Y5 antagonists, such as 152,804, GW-569180A, GW-594884A, GW-587081X, GW-548118X, FR235,208, FR226928, ER 240662, FR252384, 1229U91, GI-264879A, CCP71683A, LY-377897, LY-366377, PD-160170, SR-120562A, SR-120819A, JCF-104, and H409/22; and those disclosed in U.S. Pat. Nos. 6,140,354, 6,191,160, 6,258,837, 6,313,298, 6,337,332, 6,329,395, 6,326,375, 6,335,345, and 6,340,683, European Patent Nos. EP-01010691, and EP-01044970, and PCT Patent Publication Nos. WO 97/19682, WO 97/20820, WO 97/20821, WO 97/20822, WO 97/20823, WO 98/27063, WO 00/107409, WO 00/185714, WO 00/185730, WO 00/64880, WO 00/68197, WO 00/69849, WO 01/09120, WO 01/14376, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/20488, WO 02/22592, WO 02/48152, WO 02/49648 and WO 02/094789; and Norman et al., J. Med. Chem. 43:4288-4312 (2000);

(k) orexin-1 receptor antagonists, such as SB-334867-A, and those disclosed in PCT Patent Application Nos. WO 01/96302, WO 01/68609, WO 02/51232, WO 02/51838, and WO 03/023561;

(l) PPARα: agonists such as described in WO 97/36579 by Glaxo, and PPARα: agonists such as beclofibrate, benzafibrate, ciprofibrate, clofibrate, etofibrate, fenofibrate, gemacabene, gemfibrozil, GW 7647, BM 170744, and LY518674; and other fibric acid derivatives, such as Atromid®), Lopid® and Tricor®, and the like;

(m) PPARγ antagonists as described in WO97/10813;

(n) serotonin reuptake inhibitors such as fluoxetine, paroxetine, and sbrtraline;

(o) growth hormone secretagogues, such as MK-0677, and growth hormone secretagogue receptor agonists/antagonists, such as NN703, hexarelin, SM-130686, CP-424,391, L-692,429 and L-163,255, and such as those disclosed in U.S. Pat. Nos. 5,536,716, and 6,358,951, U.S. Patent Application Nos. 2002/049196 and 2002/022637, and PCT Application Nos. WO 01/56592 and WO 02/32888;

(p) cannabinoid receptor ligands, such as cannabinoid CB₁ receptor antagonists or inverse agonists, such as rimonabant (Sanofi Synthelabo), and SR-147778 and SR 141716A (Sanofi Synthelabo), SLV-319 (Solvay), BAY 65-2520 (Bayer), and those disclosed in U.S. Pat. Nos. 5,532,237, 4,973,587, 5,013,837, 5,081,122, 5,112,820, 5,292,736, 5,624,941,6,028,084, PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, WO 01/64632, WO 01/64633, WO 01/64634, W002/076949, WO 03/0060007, and WO 03/007887; and EPO Application No. EP-658546, EP-656354, EP-576357;

(q) protein tyrosine phosphatase-1B (PTP-1B) inhibitors; and

(r) anti-obesity agents, such as (1) melanin-concentrating hormone (MCH) receptor antagonists, such as those disclosed in WO 01/21577 and WO 01/21169; (2) melanin-concentrating hormone 1 receptor (MCH1R) antagonists, such as T-226296 (Takeda), SNP-7941, and those disclosed in PCT Patent Application Nos. WO 01/82925, WO 01/87834, WO 02/051809, WO 02/06245, WO 02/04433, WO 02/076929, WO 02/076947, WO 02/51809, WO 02/083134, WO 02/094799, and WO 03/004027, and Japanese Patent Application No. JP 13226269; (3) melanin-concentrating hormone 2 receptor (MCH2R) agonist/antagonists; (4) serotonin reuptake inhibitors such as fluoxetine, paroxetine, and sertraline, and those disclosed in U.S. patent application Ser. No. 6,365,633, and PCT Patent Application Nos. WO 01/27060 and WO 01/162341; (5) melanocortin agonists, such as Melanotan II or those described in WO 99/64002 and WO 00/74679; (6) other Mc4r (melanocortin 4 receptor) agonists, such as CHIR86036 (Chiron), ME-10142, and ME-10145 (Melacure), and those disclosed in PCT Application Nos. WO 01/991752, WO 01/74844, WO 02/12166, WO 02/11715, and WO 02/12178; (7) 5HT-2 agonists; (8) 5HT2C (serotonin receptor 2C) agonists, such as BVT933, DPCA37215, WAY161503, R-1065, IK264, and PNU 22394, and those disclosed in U.S. Pat. No. 3,914,250, and PCT Application Nos. WO 02/36596, WO 02/48124, WO 02/10169, WO 01/66548, WO 02/44152, WO 02/51844, WO 02/40456, and WO 02/40457; (9) galanin antagonists; (10) CCK agonists; (11) CCK-A (cholecystokinin-A) agonists, such as AR-R 15849, GI 181771, JMV-180, A-71378, A-71623 and SR146131, and those discribed in U.S. Pat. No. 5,739,106; (12) GLP-1 (glucagon like peptide 1 agonists; (13) corticotropin-releasing hormone agonists; (14) histamine receptor-3 (H3) modulators; (15) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, GT2394 (Gliatech), A 331440, and those described and disclosed in PCT Application No. WO 02/15905, and O-[3-(1H-imidazol-4-yl)propanol]-carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm.(Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)); (16) 11β-hydroxy steroid dehydrogenase-1 inhibitors (11β-HSD-1), such as BVT 3498, BVT 2733, and those compounds disclosed in WO 01/90091, WO 01/90090, WO 01/90092; (17) PDE (phosphodiesterase) inhibitors, such as theophylline, pentoxifylline, zaprinast, sildenafil, amrinone, milrinone, cilostamide, rolipram, and cilomilast; (18) phosphodiesterase-3B (PDE3B) inhibitors; (19) NE (norepinephrine) transport inhibitors, such as GW 320659, despiramine, talsupram, and nomifensine; (20) ghrelin receptor antagonists, such as those disclosed in PCT Application Nos. WO 01/87335, and WO 02/08250; (21) leptin, including recombinant human leptin (PEG-OB, Hoffman La Roche) and recombinant methionyl human leptin (Amgen); (22) leptin derivatives, such as those disclosed in U.S. Pat. Nos. 5,552,524, 5,552,523, 5,552,522, 5,521,283, and PCT International Publication Nos. WO 96/23513, WO 96/23514, WO 96/23515, WO 96/23516, WO 96/23517, WO 96/23518, WO 96/23519, and WO 96/23520; (23) BRS3 (bombesin receptor subtype 3) agonists; (24) CNTF (Ciliary neurotrophic factors), such as GI-181771 (Glaxo-SmithKline), SR146131 (Sanofi Synthelabo), butabindide, PD170,292, and PD 149164 (Pfizer); (25) CNTF derivatives, such as axokine (Regeneron), and those disclosed in PCT Application Nos. WO 94/09134, WO 98/22128, and WO 99/43813; (26) monoamine reuptake inhibitors, such as sibutramine (Meridia®/Reductil®), and those disclosed in U.S. Pat. Nos. 4,746,680, 4,806,570, and 5,436,272, and U.S. Patent Publication No. 2002/0006964, WO 01/27068, and WO 01/62341; (27) UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid, and those disclosed in PCT Patent Application No. WO 99/00123; (28) thyroid hormone β agonists, such as KB-2611 (KaroBioBMS), and those disclosed in PCT Application No. WO 02/15845, and Japanese Patent Application No. JP 2000256190; (29) FAS (fatty acid synthase) inhibitors, such as Cerulenin and C75; (30) DGAT1 (diacylglycerol acyltransferase 1) inhibitors; (31) DGAT2 (diacylglycerol acyltransferase 2) inhibitors; (32) ACC2 (acetyl-CoA carboxylase-2) inhibitors; (33) glucocorticoid antagonists; (34) acyl-estrogens, such as oleoyl-estrone, disclosed in del Mar-Grasa, M. et al., Obesity Research, 9:202-9 (2001); (35) dipeptidyl peptidase IV (DP-IV) inhibitors, such as isoleucine thiazolidide, valine pyrrolidide, NVP-DPP728, LAF237, P32/98P93/01, TSL 225, TMC-2A/2B/2C, FE 999011, P9310/K364, VIP 0177, SDZ 274-444; and the compounds disclosed in WO 03/004498, WO 03/004496, EP 1 258 476, WO 02/083128, WO 02/062764, WO 03/000250, WO 03/002530, WO 03/002531, WO 03/002553, WO 03/002593, WO 03/000180, and WO 03/000181; NVP-DPP728; P32/98; LAF 237, TSL 225, valine pyrrolidide, TMC-2A/2B/2C, CD-26 inhibitors, FE 999011, P9310/K364, VIP 0177, DPP4, SDZ 274-444; and the compounds disclosed in WO 03/004498; WO 03/004496; EP 1 258 476; WO 02/083128; WO 02/062764; WO 03/000250; WO 03/002530; WO 03/002531; WO 03/002553; WO 03/002593; WO 03/000180; and WO 03/000181; (36) fatty acid transporter inhibitors; (37) dicarboxylate transporter inhibitors; (38) glucose transporter inhibitors; (39) phosphate transporter inhibitors; (40) Topiramate (Topimax®); (41) 5HT (serotonin) transporter inhibitors, such as paroxetine, fluoxetine, fluvoxamine, sertraline, and imipramine; (42) opioid antagonists, such as nalmefene (Revex®), 3-methoxynaltrexone, naloxone, and naltrexone; and those disclosed in WO 00/21509; (43) Mc3r (melanocortin 3 receptor) agonists; (44) phytopharm compound 57 (CP 644,673); (45) FAS (fatty acid synthase) inhibitors, such as Cerulenin and C75; (46) SCD-1 (stearoyl-CoA desaturase-1) inhibitors; and the like; (47) aminorex; (48) amphechloral; (49) amphetamine; (50) benzphetamine; (51) chlorphentermine; (52) clobenzorex; (53) cloforex; (54) clominorex; (55) clortermine; (56) cyclexedrine; (57) dextroamphetamine; (58) diethylpropion; (59) diphemethoxidine, (60) N-ethylamphetamine; (61) fenbutrazate; (62) fenisorex; (63) fenproporex; (64) fludorex; (65) fluminorex; (66) furfurylmethylamphetamine; (67) levamfetamine; (68) levophacetoperane; (69) mazindol; (70) mefenorex; (71) metamfepramone; (72) methamphetamine; (norpseudoephedrine; (73) pentorex; (74) phendimetrazine; (75) phenmetrazine; (76) phenylpropanolamine; (77) picilorex; and (78) zonisamide; (79) PYY, PYY3-36, and PYY agonists such as those disclosed in WO 03/026591; and the like;

(s) lipid lowering agents such as (1) CETP inhibitors such as JTT 705, torcetrapib, CP 532,632, BAY63-2149, SC 591, SC 795, and the like; (2) squalene synthetase inhibitors; (3) FXR receptor modulators such as GW 4064, SR 103912, and the like; (4) LXR receptor such as GW 3965, T9013137, and XTCO179628, and the like; (5) lipoprotein synthesis inhibitors such as niacin; (6) renin angiotensin system inhibitors; (7) PPAR δ partial agonists; (8) bile acid reabsorption inhibitors, such as BARI 1453, SC435, PHA384640, S8921, AZD7706, and the like; (9) triglyceride synthesis inhibitors; (10) microsomal triglyceride transport (MTTP) inhibitors, such as inplitapide, LAB687, and CP346086, and the like; (11) transcription modulators; (12) squalene epoxidase inhibitors; (13) low density lipoprotein (LDL) receptor inducers; (14) platelet aggregation inhibitors; (15) 5-LO or FLAP inhibitors; and (16) niacin receptor agonists;

(t) anti-diabetic agents such as (1) meglitinides such as repaglinide, and nateglinide, and the like; (2) alpha-amylase inhibitors such as tendamistat, trestatin, and Al-3688, and the like; (3) insulin secreatagogues such as linogliride; and A-4166, and the like; (4) fatty acid oxidation inhibitors, such as clomoxir, and etomoxir, and the like; (5) A2 antagonists, such as midaglizole; isaglidole; deriglidole; idazoxan; earoxan; and fluparoxan, and the like; (6) non-thiazolidinediones such as JT-501, and farglitazar (GW-2570/GI-262579), and the like; (7) PPARα/γ dual agonists such as CLX-0940, GW-1536, GW1929, GW-2433, KRP-297, L-796449, LR-90, SB 219994, and MK-767, and the like; (8) other insulin sensitizing drugs; and (9) VPAC2 receptor agonists; and

(u) anti-hypertensive agents such as (1) diuretics, such as thiazides, including chlorthalidone, chlorthiazide, dichlorophenamide, hydroflumethiazide, indapamnide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (4) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (5) neutral endopeptidase inhibitors such as omapatrilat, cadoxatril and ecadotril, fosidotril, sampatrilat, AVE7688, ER4030, and the like; (6) endothelin antagonists such as tezosentan, A308165, and YM62899, and the like; (7) vasodilators such as hydralazine, clonidine, minoxidil, and nicotinyl alcohol, and the like; (8) angiotensin II receptor antagonists such as candesartan, eprosartan, irbesartan, losartan, pratosartan, tasosartan, telmisartan, valsartan, and EXP-3137, F16828K, and RNH6270, and the like; (9) α/β adrenergic blockers as nipradilol, arotinolol and amosulalol, and the like; (10) alpha 1 blockers, such as terazosin, urapidil, prazosin, bunazosin, trimazosin, doxazosin, naftopidil, indoramin, WHIP 164, and XEN010, and the like; (11) alpha 2 agonists such as lofexidine, tiamenidine, moxonidine, rilmenidine and guanobenz, and the like; and (12) aldosterone inhibitors, and the like.

Examples of other anti-obesity agents that can be employed in combination with a compound of Formula I are disclosed in “Patent focus on new anti-obesity agents,” Exp. Opin. Ther. Patents, 10: 819-831 (2000); “Novel anti-obesity drugs,” Exp. Opin. Invest. Drugs, 9: 1317-1326 (2000); and “Recent advances in feeding suppressing agents: potential therapeutic strategy for the treatment of obesity, Exp. Opin. Ther. Patents, 11: 1677-1692 (2001). The role of neuropeptide Y in obesity is discussed in Exp. Opin. Invest. Drugs, 9: 1327-1346 (2000). Cannabinoid receptor ligands are discussed in Exp. Opin. Invest. Drugs, 9: 1553-1571 (2000). Various pharmacological approaches for the treatment of obesity is discussed in J-A Fernandez-Lopez, Drugs: 62: 915-944 (2002); in H. Bays, et al., “Anti-obesity drug development,” Exp. Opin. Invest. Drugs, 11: 1189-1204 (2002); and in D. Spanswick, et al., “Emerging Anti-obesity Drugs,” Exp. Opin. Emerging Drugs, 8(1): 217-237 (2003).

Examples of other active ingredients that may be combined with a compound of Formula I for the treatment or prevention of male or female sexual dysfunction, in particular, male erectile dysfunction, either administered separately or in the same pharmaceutical compositions, include, but are not limited to (a) type V cyclic-GMP-specific phosphodiesterase (PDE-V) inhibitors, including sildenafil and (6R, 12aR)-2,3,6,7,12,12a-hexahydro-2-methyl-6-(3,4-methylenedioxyphenyl)-pyrazino[2′,1′: 6,1]pyrido[3,4-b]indole-1,4-dione (IC-351); (b) alpha-adrenergic receptor antagonists, including phentolamine and yohimbine or pharmaceutically acceptable salts thereof; (c) dopamine receptor agonists, such as apomorphine or pharmaceutically acceptable salts thereof; and (d) nitric oxide (NO) donors.

The instant invention also includes administration of a single pharmaceutical dosage formulation which contains both the MC-4R agonist in combination with a second active ingredient, as well as administration of each active agent in its own separate pharmaceutical dosage formulation. Where separate dosage formulations are used, the individual components of the composition can be administered at essentially the same time, i.e., concurrently, or at separately staggered times, i.e. sequentially prior to or subsequent to the administration of the other component of the composition. The instant invention is therefore to be understood to include all such regimes of simultaneous or alternating treatment, and the terms “administration” and “administering” are to be interpreted accordingly. Administration in these various ways are suitable for the present compositions as long as the beneficial pharmaceutical effect of the combination of the MC-4R agonist and the second active ingredient is realized by the subject at substantially the same time. Such beneficial effect is preferably achieved when the target blood level concentrations of each active ingredient are maintained at substantially the same time. It is preferred that the combination of the MC-4R agonist and the second active ingredient be co-administered concurrently on a once-a-day dosing schedule; however, varying dosing schedules, such as the MC-4R agonist once a day and the second active ingredient once, twice or more times per day, is also encompassed herein. A single oral dosage formulation comprised of both a MC-4R agonist and a second active ingredient is preferred. A single dosage formulation will provide convenience for the subject, which is an important consideration especially for subjects with diabetes or obese subjects who may be in need of multiple medications.

The above combinations include combinations of a composition of the present invention not only with one other active compound, but also with two or more other active compounds. Non-limiting examples include combinations of the compositions of the present invention with one, two or more active compounds selected from lipid-lowering agents, and anti-hypertensive agents. Combinations of the compositions of the present invention with one, two or more active compounds selected from lipid lowering agents, and anti-diabetic agents are useful to treat, control or prevent metabolic syndrome. In particular, compositions comprising an anti-obesity agent, such as a melanocortin-4 receptor agonist, an anti-hypertensive agent, in addition to an anti-diabetic agent and/or a lipid lowering agent will be useful to synergistically treat, control or prevent metabolic syndrome.

The compounds in the combinations of the present invention may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of the melanocortin-4 receptor agonist, or a pharmaceutically acceptable salt or ester thereof, and a pharmaceutically acceptable carrier or diluent in a first unit dosage form, and a second unit dosage form comprising a prophylactically or therapeutically effective amount of the second active ingredient or drug, or a pharmaceutically acceptable salt or ester thereof, and a pharmaceutically acceptable carrier or diluent in a second unit dosage form.

In one embodiment, the kit further comprises a container. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

Pharmaceutical Compositions

Another aspect of the present invention provides pharmaceutical compositions which comprises a compound of Formula I and a pharmaceutically acceptable carrier. The pharmaceutical compositions of the present invention comprise a compound of Formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Based on their ease of administration, tablets and capsules represent the most advantageous oral dosage unit form in which case solid pharmaceutical carriers are employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Preparation of Compounds of the Invention

The compounds of structural formula I of the present invention can be prepared according to the procedures of the following Schemes and Examples, using appropriate materials and are further exemplified by the following specific examples. Moreover, by utilizing the procedures described in detail in PCT International Application Publications WO 02/068387 (6 Sep. 2002) and WO 002/068388 (6 Sep. 2002), which are incorporated by reference herein in their entirety, in conjunction with the disclosure contained herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those described previously hereinabove. The free amine bases corresponding to the isolated salts can be generated by neutralization with a suitable base, such as aqueous sodium hydrogencarbonate, sodium carbonate, sodium hydroxide, and potassium hydroxide, and extraction of the liberated amine free base into an organic solvent followed by evaporation. The amine free base isolated in this manner can be further converted into another pharmaceutically acceptable salt by dissolution in an organic solvent followed by addition of the appropriate acid and subsequent evaporation, precipitation, or crystallization. All temperatures are degrees Celsius unless otherwise noted. Mass spectra (MS) were measured by electron-spray ion-mass spectroscopy.

The phrase “standard peptide coupling reaction conditions” means coupling a carboxylic acid with an amine using an acid activating agent such as EDC, DCC, and BOP in an inert solvent such as dichloromethane in the presence of a catalyst such as HOBT. The use of protecting groups for the amine and carboxylic acid functionalities to facilitate the desired reaction and minimize undesired reactions is well documented. Conditions required to remove protecting groups are found in standard textbooks such as Greene, T, and Wuts, P. G. M., Protective Groups in Organic Synthesis, John Wiley & Sons, Inc., New York, N.Y., 1991. CBZ and BOC are commonly used protecting groups in organic synthesis, and their removal conditions are known to those skilled in the art. For example, CBZ may be removed by catalytic hydrogenation in the presence of a noble metal or its oxide such as palladium on activated carbon in a protic solvent such as methanol or ethanol. In cases where catalytic hydrogenation is contraindicated due to the presence of other potentially reactive functionalities, removal of CBZ groups can also be achieved by treatment with a solution of hydrogen bromide in acetic acid or by treatment with a mixture of TFA and dimethylsulfide. Removal of BOC protecting groups is carried out with a strong acid, such as trifluoroacetic acid, hydrochloric acid, or hydrogen chloride gas, in a solvent such as methylene chloride, methanol, or ethyl acetate.

Abbreviations Used in the Description of the Preparation of the Compounds of the Present Invention:

BOC (boc) is t-butyloxycarbonyl, BOP is benzotriazol-1-yloxytris(dimethyl-amino)phosphonium hexafluorophosphate, Bu is butyl, calc. is calculated, CBZ (Cbz) is benzyloxycarbonyl, c-hex is cyclohexyl, c-pen is cyclopentyl, c-pro is cyclopropyl, DIEA is diisopropylethylamine, DIPEA is N,N-diisopropylethylamine, DMAP is 4-dimethylaminopyridine, DMF is N,N-dimethylformamide, DMSO is dimethyl sulfoxide, EDC is 1-(3-dimethyl-aminopropyl)3-ethylcarbodiimide HCl, eq. is equivalent(s), ES-MS is electron spray ion-mass spectroscopy, Et is ethyl, EtOAc is ethyl acetate, hr is hour, min is minutes, HATU is O-(7-azabenzotriazol-1-yl)-1,1,3,3-tetramethyluronium hexafluorophosphate, HOAt or HOAt is 1-hydroxy-7-azabenzotriazole, HOBt is 1-hydroxybenzotriazole hydrate, HPLC is high performance liquid chromatography, IPA is isopropyl alcohol, LDA is lithium diisopropylamide, LiHMDS is lithium hexamethyl disilazane, MC-xR is melanocortin receptor (x being a number), Me is methyl, MF is molecular formula, MPLC is medium pressure liquid chromatography, MS is mass spectrum, Ms is methanesulfonyl, MTBE is tert-butyl methyl ether, NMM is N-methylmorpholine, OTf is trifluoromethanesulfonyl, Ph is phenyl, Phe is phenylalanine, Pr is propyl, prep. is prepared, PyBOP is benzotriazol-1-yloxytripyrrolidine phosphonium hexafluorophosphate, RT (rt) is room temperature, (S)-2-methyl-CBS-oxazaborolidine is (S)-tetrahydro-1-methyl-3,3-dipheyl-1H,3H-pyrrolo[1,2-c][1,3,2]oxazaborole, TEA is triethyl amine, TFA is trifluoroacetic acid, TBF is tetrahydrofuran, and TLC is thin-layer chromatography.

Reaction Schemes A-F illustrate methods employed in the synthesis of the compounds of the present invention of structural formula I. All substituents are as defined above unless indicated otherwise.

Reaction Scheme A illustrates a key step in the synthesis of the novel compounds of structural formula I of the present invention. As shown in reaction Scheme A, the reaction of a piperidine derivative of type 1 with a carboxylic acid derivative of formula 2 affords a title compound of structural formula I. The amide bond coupling reaction illustrated in reaction Scheme A is conducted in an appropriate inert solvent such as DMW, methylene chloride or the like and may be performed with a variety of reagents suitable for aride coupling reactions such as HATU, EDC or PyBOP. Preferred conditions for the amide bond coupling reaction shown in reaction Scheme A are known to those skilled in organic synthesis. Such modifications may include, but are not limited to, the use of basic reagents such as TEA, DIPEA, or NMM, or the addition of an additive such as HOAt or HOBt. Alternatively, 4-substituted piperidines of formula 1 may be treated with an active ester or acid chloride derived from carboxylic acid 2 which also affords compounds of structural formula I. The amide bond coupling shown in reaction Scheme A is usually conducted at a temperature between 0° C. and room temperature, occasionally at elevated temperatures, and the coupling reaction is typically conducted for periods of 1 to 24 hours.

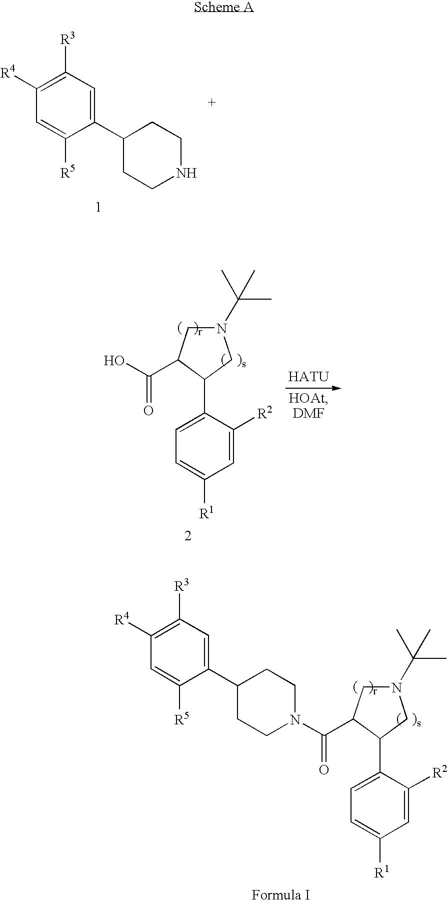

The synthesis of carboxylic acids of general formula 2 utilized in the amide bond coupling reaction in Scheme A was previously described in WO 02/068387 (6 Sep. 2002) and WO 02/068388 (6 Sep. 2002). Reaction Schemes B-F illustrate methods for the synthesis of the carboxylic acids of general formula 2 that are utilized in the amide bond coupling reaction shown in reaction Scheme A. These schemes also feature methods for modification or elaboration of compounds of general formula I.

Reaction Scheme B illustrates a strategy for the synthesis of compounds of general formula 2 wherein the values of r and s are selected such that the resulting heterocycle is a 3-aryl-4-pyrrolidine carboxylic acid derivative 8. The preferred method for the synthesis of compounds of general formula 8 involves the azomethine ylid 3+2 cycloaddition reaction of an azomethine ylid precursor of general formula 4 and a substituted cinnamic ester 3. The azomethine cycloaddition reaction of 3 and 4 affords the 3,4-disubstituted pyrrolidine 5, and the stereochemical relationship of the substituents on the newly formed pyrrolidine ring is determined by the stereochemistry of the double bond in the cinnamate ester 3. Thus the trans ester 3 affords a trans 3,4-disubstituted pyrrolidine of formula 5. The corresponding cis cinnamate ester affords a cis 3,4-disubstituted pyrrolidine of general formula 5. Cis or trans 3-arylpyrrolidine-4-carboxylic esters of general formula 5 may be resolved to afford enantiomerically pure compounds using a method such as resolution by crystallization of the diastereoisomeric salts derived from 5 and a chiral carboxylic acid, or directly by the use of chiral stationary phase liquid chromatography columns. Reaction Scheme B illustrates the case where a trans cinnamic ester 3 is converted to a trans 3,4-disubstituted pyrrolidine 5 and its subsequent resolution affords the enantiomerically pure trans pyrrolidine esters 6 and 7. Finally, the esters of general formula 5 (or their pure enantiomers 6 and 7) are hydrolyzed to the corresponding amino acid hydrochlorides of general formula 8 as shown at the bottom of reaction Scheme B.

Amino acids of general formula 8 are zwitterionic. Therefore it is in some cases difficult to achieve efficient separation and purification of these compounds from aqueous reactions or workups. In these cases it is preferred to effect the hydrolysis using a reagent such potassium trimethylsilanolate in diethyl ether. Under these conditions the potassium salt of the carboxylic acid is produced which affords an easily isolated precipitate in ether. The resulting salt is then converted to the corresponding amino acid hydrochloride by treatment with excess hydrogen chloride in a suitable solvent such as ethyl acetate. Alternatively, esters such as 5 may be converted directly to the amino acid hydrochlorides 8 under acidic hydrolysis conditions. The hydrolysis of the ester 5 is achieved by prolonged reaction with concentrated hydrochloric acid at an elevated temperature. For example, this reaction may be conducted in 8 M hydrochloric acid at reflux overnight. The reaction mixture is then cooled and evaporated in vacuo to afford the amino acid hydrochloride 8. The amino acid hydrochlorides of general formula 8 correspond to an amino acid hydrochloride of general formula 2 wherein both r and s are 1 and may be employed directly in the amide bond coupling step illustrated in reaction Scheme A to produce the compounds of the present invention of structural formula I.

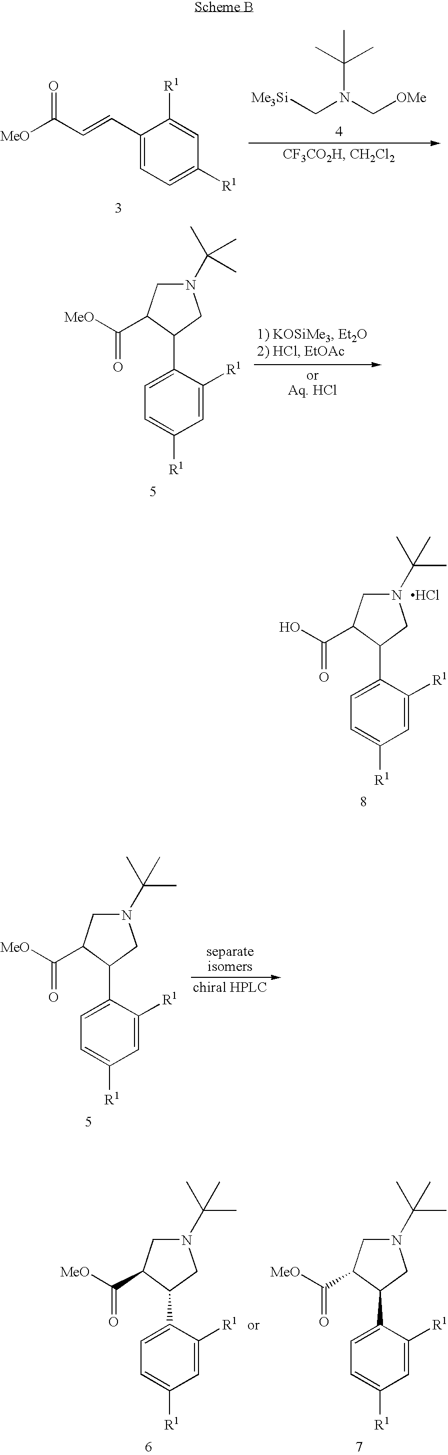

When it is desired to prepare individual enantiomers of the novel title compounds of structural formula I, it is possible to perform a resolution of the compounds of structural formula I using one of the methods known in the art of organic synthesis. For instance, enantiomerically pure compounds (I) may be prepared by crystallization of diastereoisomeric salts formed from the racemic compounds of structural formula I and an optically active carboxylic acid. The two diastereoisomeric salts are separated from each other by fractional crystallization, then the enantiomerically pure compounds of structural formula I are regenerated by treatment of the purified salts with a base. Alternatively, racemic compounds of structural formula I may be resolved by preparative HPLC using commercially available chiral stationary phase columns. Another strategy for the preparation of enantiomerically pure compounds of structural formula I involves preparing enantiomerically pure compounds of general formula 2 prior to their use in the amide bond forming reaction outlined in reaction Scheme A. Racernic compounds of general formula 2, or intermediates used to prepare compounds of formula 2 as described in the previous reaction Schemes (i.e. acid 8, or ester 5) may also be resolved using the classical methods previously discussed.

Scheme C illustrates the preparation of azomethine precursors of formula 4 starting with amines of general formula 9. Reaction of the amine of formula 9 with chloromethyltrimethylsilane at high temperature and in the absence of solvent affords the N-trimethylsilylmethyl-substituted amine of general formula 10. Subsequent reaction of 10 with aqueous formaldehyde in the presence of methanol and a base such as potassium carbonate then affords the generalized ylid precursor 4 which can be utilized in the cycloaddition reactions discussed above.

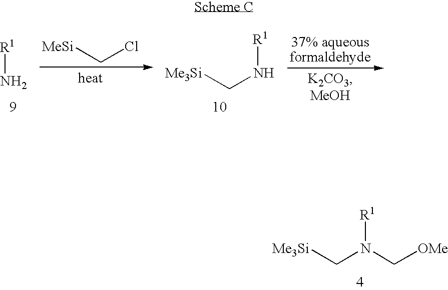

Scheme D illustrates a strategy for the synthesis of compounds of general formula 2 wherein r is 1 or 2 and s is 1. The synthesis involves the stereoselective reduction of the ketone of compound 11 to give the alcohol 12, and the displacement of the chloride with tert-butyl amine to give compound 13. The nitrogen may then be alkylated via a Michael addition to acrylonitrile, or via reaction with a leaving group substituted alkyl nitrile, such as a bromo butyro nitrile or a bromo propionitrile to give compound 14. Compound 14 is then cyclized to give compound 15, and the nitrile of compound 15 may be hydrolyzed to give the pyrrolidine acid 16. 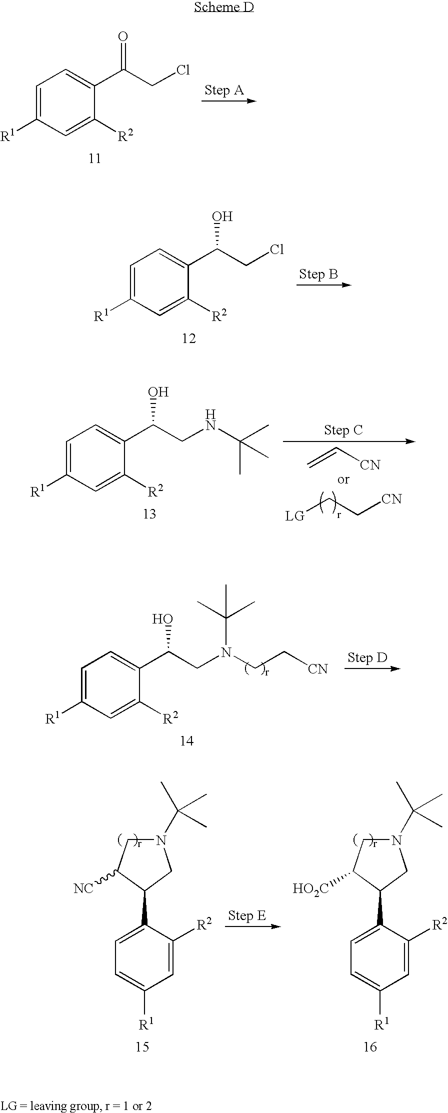
Alternatively, the nitrile of compound 15 maybe converted to pyrrolidine acid 16 by conversion of nitrile 15 to methyl ester 18, via amide 17, and subsequent hydrolysis to give acid 16 as shown in Scheme E.

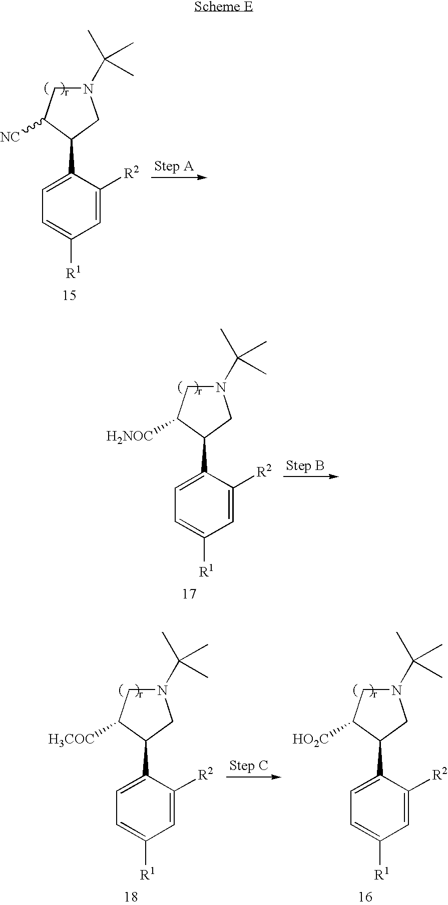

Reaction Scheme F illustrates a preferred method for elaboration of the aryl piperdine substituent to generate compounds of structural formula I wherein R⁵ is —(CH₂)ₙ(NR⁶)C(O)R⁶, wherein methylene (CH₂)ₙ is optionally substituted with one to two substituents selected from the group consisting of: halogen, hydroxy, oxo, C₁₋₄ alkyl, trifluoromethyl, and C₁₋₄ alkoxy. Treatment of enol triflate 19 (prepared as described in: Rohr, M.; Chayer, S.; Garrido, F.; Mann, A.; Taddei, M.; Wermuth, C.-G. Heterocycles 1996, 43, 2131) with bis(pinacolato)diboron reagent in the presence of a suitable palladium (II) catalyst such as [1,1′-Bis(diphenylphosphino)-ferrocene]dichloropalladium (II) (Pd(dppf)Cl₂) and potassium acetate in a polar, inert organic solvent such as methyl sulfoxide at about 80° C. under an inert atmosphere for a period of 6-24 hours provides the vinyl dioxaborolane 20. The synthesis of borolane 20 was previously disclosed in WO 02/068388. Triflate 23 can be further elaborated by reaction with borolane 20 in the presence of a palladium catalyst such as tetrakis(triphenylphosphine) palladium(0) and aqueous sodium carbonate in a degassed mixture of ethanol and toluene with heating to give the coupled 4-aryl tetrahydropyridine product 24. Triflate 23 is prepared by the treatment of phenol 21 with propionic chloride in the presence of aluminum chloride to give substituted phenol 22. Phenol 21 may also be treated with other acid chlorides to give the appropriate ortho ketone substituent. Phenol 22 can be converted to the triflate using a triflating reagent such as triflic anhydride in the presence of a tertiary amine such as triethylamine and a catalytic amount of 4-(dimethylamino)pyridine in an inert solvent such as methylene chloride at low temperature. Reduction of the double bond of intermediate 24 can be effected by treatment with hydrogen at atmospheric pressure and a noble metal catalyst on carbon such as platinum (IV) oxide or palladium (0) in an inert solvent such as ethanol, ethyl acetate, acetic acid or mixtures thereof. The carbonyl moiety can be reduced in the same conditions or is further reduced by treatment with a noble reducing agent such as sodium borohydride or sodium cyanoborohydride thereof to afford the 4-arylpiperidine 25. Conversion of the alcohol to the amide and removal of the tert-butyloxycarbonyl protecting group by treatment of piperidine 25 with sulfuric acid and acetonitrile provides the amine salt of 26. Compound 26 may be coupled with acid 2_by employing any of the large number of amide bond-forming reagents as described in Scheme A to form compound 27 (the structural formula I wherein R⁵ is —CH(R)NHCOCH₃; R¹, R², R³, and R⁴ are as previously described; and R is C₁₋₄ alkyl, as shown in Scheme F.

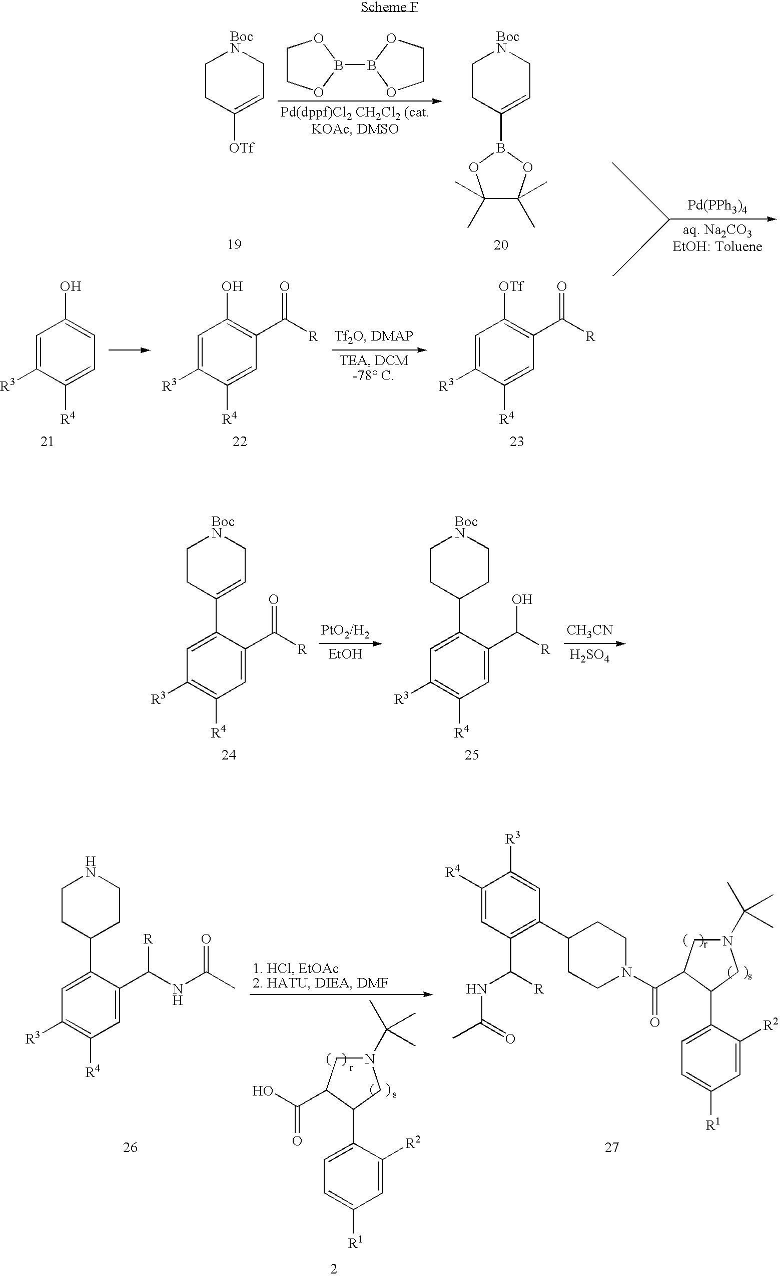

Reaction Schemes G and H illustrate preferred methods for the synthesis of pyrrolidine acid intermediates (G-6, G-7) and piperidine acid intermediates (H-5) useful to prepare compounds of structural formula I.

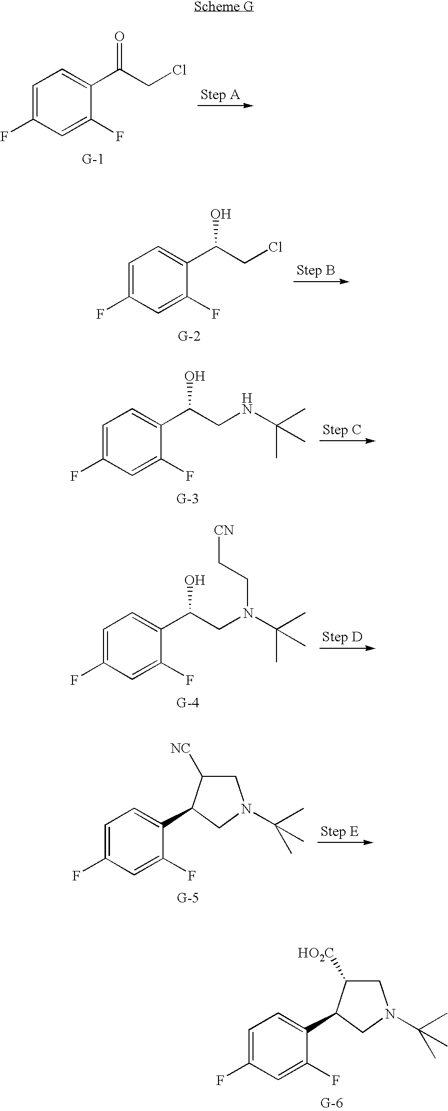

### Preparation of Intermediate (3S,4R)-1-tert-butyl-4-(2,4-difluorophenyl)pyrrolidine-3-carboxylic acid G-6

Step A: A solution of (S)-tetrahydro-1-methyl-3,3-diphenyl-1H,3H-pyrrolo[1,2-c][1,3,2]oxazaborole (131 mL, 1 M in toluene), borane-N,N-diethylaniline (46.36 L) in MTBE (10 L) was heated to 38-42° C., then a solution of 2-chloro-2′,4′-di-fluoro-acetophenone G-1 (4891 g) in MTBE (16 L) was added over 10 hr. The homogeneous solution was stirred at 40° C. for one hour, then allowed to cooled to 18° C. and stirred overnight. Methanol (2.3 L) was added over 60 min, while maintaining the temperature at <20° C. with cooling. The reaction mixture was stirred 30 min, then 5 N aq HCl (10 L) was added over 30 min, while maintaining the temperature at 22-25° C. with cooling. After stirring 30 min, the phases were separated, and the organic phase was washed with saturated aqueous NaCl, then concentrated in vacuo to obtain a solution of compound G-2.

Step B: Compound G-2 in the MTBE solution from Step A (5040 g, 98 wt %, 25.67 mol) was diluted with methanol (5 L), then tert-butylamine (25 L) was added. The mixture was cooled to 25° C., solid NaOH pellets (1048 g) were added, and the resulting reaction mixture was stirred and warmed to reflux. After 12-20 hr at reflux, the mixture was concentrated in vacuo to ⅓ volume, then water (5 L) and MTBE (20 L) were added. The phases were separated and the aqueous phase was re-extracted with MTBE (2×2 L). The combined extracts were washed with saturated aqueous NaCl (1 L), and then concentrated in vacuo. Heptane (40 L) was added and the concentration was continued to bring the volume to 20 L. The mixture was heated to ˜90° C. to dissolve all solids, then allowed to cool to 22° C. to crystallize over 4 hr. The mixture was cooled to 0° C., stirred 12-15 hr, then filtered. The resulting filtrate was washed with cold heptane (2×5 L), then dried in vacuo at 35° C. to obtain compound G-3.

Step C: A mixture of compound G-3 (5.205 kg, 99.9%, 22.68 mol) and acrylonitrile (26.9 L, 408 mol) was heated to reflux (˜77° C.) under nitrogen atmosphere. After heating for 20 h (˜90% conversion), one equivalent each of ethanol (1.32 L, 22.68 mol) and formamide (0.9 L, 22.68 mol) was added and heating was continued for 12 h. After cooling to 22° C., the solution was concentrated to 12 L by distillation (80-90 torr at 20-22° C. pot temperature), and the resulting residue was diluted with isopropyl acetate (22 L) and re-concentrated (55-75 torr and 22-27° C. pot temperature). This was repeated. Then the residue was diluted with isopropyl acetate to a total volume of 34 L, and the supernatant was filtered using a 10-15 μm porosity filter. The filter cake was washed with isopropyl acetate, and the filtrate was diluted with a total of 24 L of isopropyl acetate. The combined filtrate (˜54 L) was washed with a solution made up of water (31.2 L), acetic acid (52 mL, 4 mol %), and saturated brine (3.1 L), followed by a 12% aqueous NaCl wash (2×34 L). The organic layer was concentrated (15-45 torr and 5-29° C.) to ˜15 L volume and flushed with 5×6 L n-heptane, during which time crystallized. The slurry was diluted with n-heptane to a volume of 23 L and stirred at 0-5° C. for 1-3 days until a concentration of 10 g/18 L was achieved, then filtered and washed with cold (5° C.) n-heptane (14 L). The wet cake was dried in vacuo at 20° C. with a nitrogen sweep to afford compound G-4.

Step D: A solution of compound G-4 (5.73 kg, 99.9%, 20.28 mol) in dry TBF (31.3 L) was cooled to −20° C., then chloro diethylphosphate (3.79 kg, 21.29 mol) was added. LiHMDS (1.35 M in TBF solution; 31.5 L, 42.58 mol) was slowly added over 1.5 h while maintaining the reaction temperature at −15° C. After stirring at −15° C for 2 h, the reaction mixture was quenched with water (50.6 L) at <15° C. and extracted with n-heptane (40.5 L) at 20° C. The organic layer was washed with 10% aq NaCl solution (52 L), and extracted with 3 N HCl solution (40.6 L, 121.8 mol) with cooling to keep the temperature <35° C. The aqueous layer (58 L) was adjusted to pH 11-12 with 50% aq NaOH (6.13 L, 116.1 mol) and extracted with n-heptane (54 L). The organic phase was washed once with 10% aq NaCl solution (26 L) and the resulting heptane solution containing compound G-5 was used in Step E.

Step E: The solution of compound G-5 (4.88 kg, 18.46 mol) in n-heptane (˜65 L total) from the Step D was solvent-switched to ethanol (˜20.6 L total). To this solution was added 50% aq NaOH (2.7 L, 51.15 mol) over 2 min with stirring. Upon addition of the NaOH, the temperature of the mixture rose from 16 to 34° C. The mixture was then heated to reflux (78-80° C.) under nitrogen for 5-6 h. After cooling to 20° C., the solution was diluted with ethanol (25.4 L) and methanol (40.6 L). The solution was then cooled to 12° C.; the pH was adjusted to apparent pH 6.8 with 96% H₂SO₄ (1.42 L, 25.6 mol maintaining the temperature at ˜20° C. The sodium sulfate slurry was filtered through a bed of Solka-Floc® (5 kg) and anhydrous powder Na₂SO₄ (4 kg), and washed with 1: 1 EtOH:MeOH (20 L). The filtrate was filtered, concentrated and solvent-switched to a 2-propanol solution (˜15 L volume). The resulting slurry was heated at reflux (˜80° C.) for 2 h, then cooled to 16° C. MTBE (30.4 L, 3 vol relative to IPA) was added to the mixture over 5 h. After stirring at 16-17° C. for 3 days, the resulting slurry was filtered and washed with 12 L 1:3 IPA:MTBE. The solids were dried in vacuo (150 torr) at 50° C. with a nitrogen sweep to give compound G-6.

Following the synthetic route in Scheme G and using the appropriate reagents, (3S,4R)-1-tert-butyl-4-(2-fluoro-4-chlorophenyl)pyrrolidine-3-carboxylic acid G-7 was prepared:

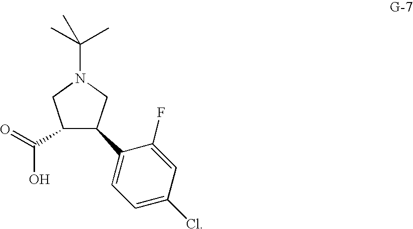

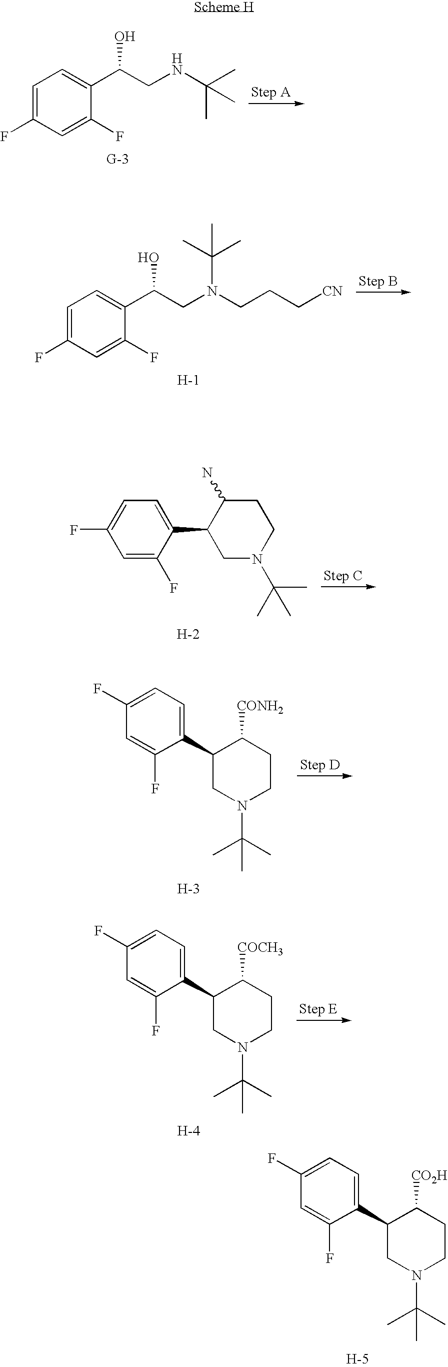

### Preparation of (3R,4R)-1-tert-butyl-3-(2,4-difluorophenyl)piperidine-4-carboxylic acid (H-5)

Step A: A mixture of compound G-3 (24 g, 0.105 mol), 4-bromobutyronitrile (42 g, 0.28 mole, 2.7 eq) K₂CO₃ (22 g, 0.16 mol, 1.52 eq) and DMF (70 mL) was heated at 50° C. for 64 hr. The reaction was quenched into water (500 mL) and extracted with ether (2×250 mL). The ether layer was extracted with 1N HCl (2×125 mL), and the resulting aqueous layer was extracted with hexanes (2×100 mL). The aqueous layer was then made basic with 5N NaOH, and extracted with ether (2×250 mL). The organic layer was washed with brine, dried with Na₂SO₄, filtered and concentrated. The residue was chromatographed (silica, 9/1 hexanes/THF then 4:1 hexanes/THF) to give compound H-1 as a colorless oil.

Step B: Compound H-1 (50 g, 0.169 mol) was dissolved in TBF (500 mL) and the solution was cooled to ˜15° C. Diethyl chlorophosphonate (25 mL, 1.74 mol, 1.03 eq) was added, followed by the dropwise addition of 1M LiHMDS in TBF (350 mL, 2.07 eq). The LiHMDS was added over 100 minutes while maintaining a reaction temperature between ˜12° C. and ˜15° C. The reaction was allowed to warm slowly to RT and aged overnight. The reaction was quenched with water and extracted twice with ether. The ether layer was washed with brine, dried with sodium sulfate, filtered and concentrated to give compound H-2.

Step C: Compound H-2 was dissolved in ethanol (150 mL), 50% NaOH (24 mL) was added and the mixture was refluxed for 5 hours. The reaction was acidified with 12 N HCl (60 mL) at which point it solidified. The mass was diluted with ethanol (50 mL) and methanol (200 mL), and filtered. The cake was washed with ethanol, and the filtrate was concentrated and flushed with isopropyl alcohol (500 mL). Additional isopropyl alcohol was added and the mixture concentrated to circa 300 mL. The slurry was filtered and the resulting cake was washed with isopropyl alcohol. The solid cakes were combined to give compound H-3.

Step D: Compound H-3 was dissolved in methanol (1L) and saturated with HCl gas. The solution was refluxed for 72 hr, then concentrated and partitioned between ether and saturated NaHCO₃ solution. The ether layer was dried with Na₂SO₄, filtered and concentrated to afford compound H-4. Additional compound H-4 was obtained from the filtrate above by similar treatment with HCl/MeOH followed by chromatography (silica 90/10/1 CH₂Cl₂/MeOH/NH₄OH).

Step E: Compound H-4 was dissolved in 6N HCl (300 mL) and the solution was refluxed for 3 hr. The solution was then concentrated and the resulting residue was dissolved in water and re-concentrated. The residue was then flushed with isopropyl alcohol (2×300 mL) and ethyl acetate (2×500 mL). The resulting slurry was stirred at room temperature for 1 hr and filtered. The resulting solid was washed with ethyl acetate and dried to give compound H-5.

### EXAMPLE 1

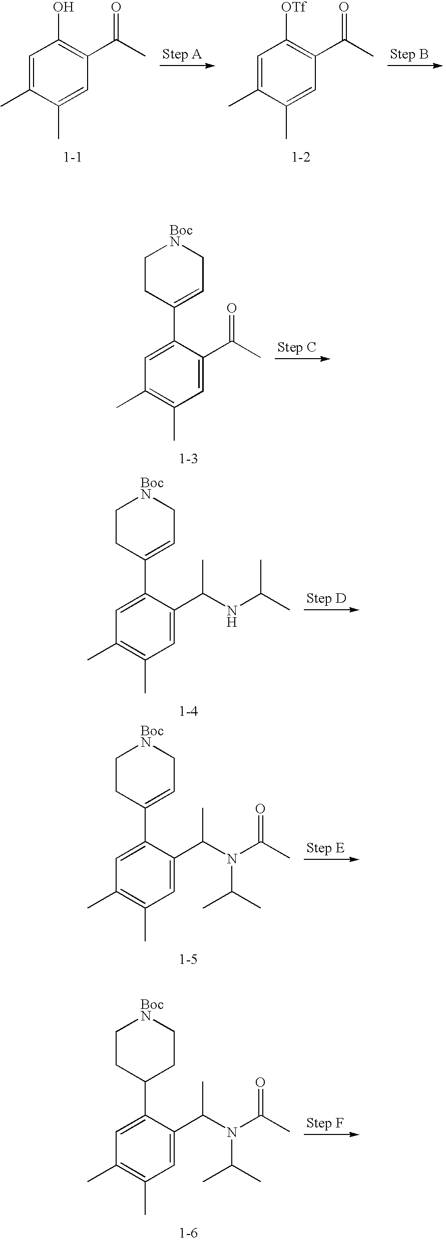

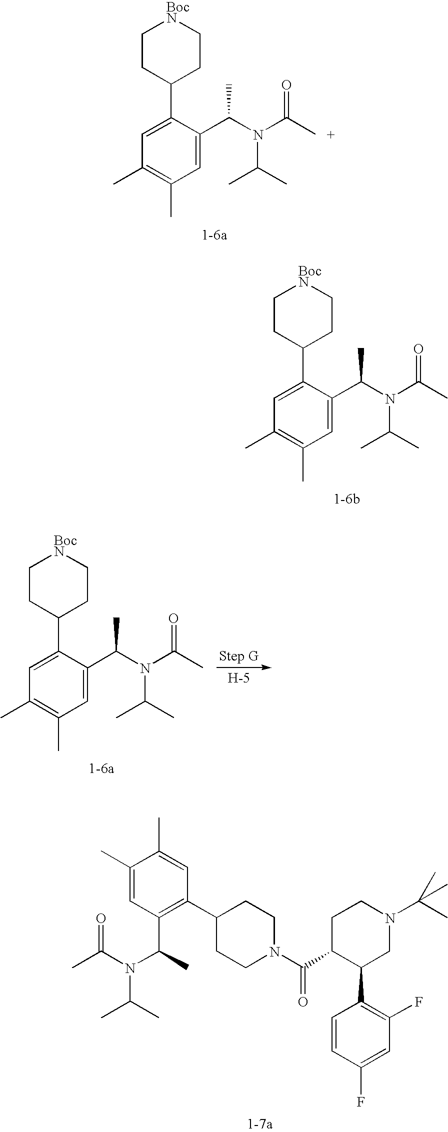

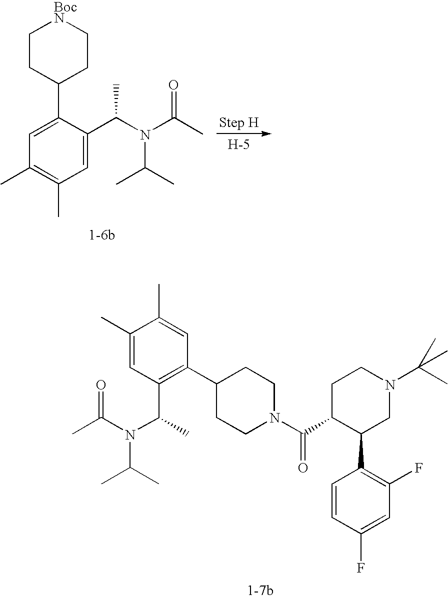

### Step A: Preparation of 4.5-dimethyl-2-propionylphenyl trifluoromethanesulfonate (1-2)

Trifluoromethanesulfonic anhydride (5.4 mL, 31.9 mmol) was added to a stirred mixture of ketone 1-1 (5.0 g, 30.4 mmol, ACROS), and pyridine (7.4 ml, 91.2 mmol) in methylene chloride (100 mL) at 0° C. The reaction mixture was allowed to slowly warmed up to RT and stirred at RT overnight. The reaction mixture was concentrated and the resulting residue was purified by MPLC eluting with 80:20 hexanes/ethyl acetate, to give 1-2 as a white solid.

### Step B: Preparation of tert-butyl 4-(4,5-dimethyl-2-propionylphenyl)-3,6-dihydropyridine-1(2H)-carboxylate (1-3)

A vigorously stirred solution of triflate 1-2 (4.45 g, 15 mmol), sodium carbonate (4.77 g, 45 mmol), tert-butyl 4-(4,4,5,5-tetramethyl-1,3,2-dioxaborolan-2-yl)-3,6-dihydropyridine-1(2H)-carboxylate (4.635 g, 15 mmol), and tetrakis-(triphenylphosphine)palladium(0) (3.479 g, 3.0 mmol) in ethanol/toluene/H₂O (60 ml/60 ml/10 ml) was degassed via three vacuum/nitrogen ingress cycles and heated to 65° C. for 3 days. The resulting solids in reaction mixture were filtered off, and the filtrate was concentrated in vacuo. Purification of the resulting crude residue by MPLC on silica gel (gradient elution: 0%-20% ethyl acetate/hexanes) afforded 1-3 as a light brown oil.

### Step C: Preparation of 1-4

To a stirred solution of ketone 1-3 (2.0 g, 6.07 mmol) in isopropyl amine (2.ml, 24.3 mmol), was added Ti(OEt)₄ (2.77 g, 12.14 mmol). After stirring at RT over weekend, methanol (50 ml) was added, followed by addition of NaBH₄ (675.6 mg, 18.2 mmol). After 30 minutes, the reaction was quenched with 1N NaOH (50 ml), and extracted three times with EtOAc. The combined organic extracts were washed with brine, dried over Na₂SO₄, filtered, and the filtrate was concentrated in vacuo to give crude product, which was used without further purification in step D.

### Step D: Preparation of 1-5

To a stirred solution of the compound 1-4 (1.6 g, 4.3 mmol) in pyridine (5 ml) was added acetic anhydride (4.1 ml, 20.1 mmol) at room temperature. The mixture was stirred at 80° C. overnight, then concentrated and purified by MPLC (gradient elution: 0% EtOAc to 40% EtOAc) to afford the desired product 1-5.

### Step E: Preparation of (1-6)

To a stirred solution of compound 1-5 (1.35 g, 3.24 mmol) in EtOH-EtOAc (50 ml/50 ml) was added Pd (OH)₂/C (500 mg). The system was degassed via two vacuum/hydrogen ingress cycles and stirred at RT in the presence of H₂ for 3 days. The catalyst was filtered off, and the filtrate was concentrated in vacuo to give the desired product 1-6 as white solid.

### Step F: Preparation of the single enantiomers of tert-butyl 4-{2-[1-(acetylamino)propyl]-5,4-dimethylphenyl]piperidine-1-carboxylate (1-6a and 1-6b)

The racemic mixture of tert-butyl 4-{2-[1-(acetylamino)propyl]-5,4-dimethylphenyl}piperidine-1-carboxylate 1-6 (640 mg) was separated using high performance liquid chromatography over ChiralCel OD 20×250 mm (˜50 mg per injunction; isocratic elution, 5% EPA in heptane; flow rate=9 mL/min; UV detector wavelength=254) afforded the titled compounds 1-6a and 1-6b as white solids.

### Step G: Preparation of the single diastereomer of N-{1-[2-(1-{[(3S,4R)-1-tert-butyl-4-(2,4-difluorophenyl)pyrrolidin-3-yl]carbonyl}piperidin-4-yl)-4-chloro-5-methylphenyl]propyl}acetamide (1-7a)

To the single enantiomer of tert-butyl 4-(2-{1-[acetylamino]ethyl}-5-chloro-4-methylphenyl)piperidine-1-carboxylate 1-6a (220 mg, 0.522 mmol) was added 4 N HCl in dioxane (10 mL) at RT. The mixture was stirred at room temperature for 60 min, then the volatiles were removed under reduced pressure. The resulting residue was dissolved in dichloromethane (5 mL), and (3S,4R)-1-tert-butyl-4-(2,4-difluorophenyl)pyrrolidine-3-carboxylic acid H-5 (180 mg, 0.539 mmol), DIEA (0.375 mL, 0.636 mmol), HOAT (73 mg, 0.539 mmol), and HATU (410 mg, 1.078 mmol) were added. The mixture was stirred at room temperature overnight. The solvents were removed under vacuum, and the resulting residue was purified by preparative TLC eluting with 10% MeOH in dichloromethane to give compound 1-7a. Compound 1-7a was isolated and converted to the HCl salt by treatment with 1N HCl in ether. Calc MW. for C₃₂H₄₂ClF₂N₃O₂: 596; Found: 597 (M+H).

### Step H: Preparation of the single diastereomer of N-{1-[2-(1-{[(3S,4R)-1-tert-butyl-4-(2,4-difluorophenyl)pyrrolidin-3-yl]carbonyl}piperidin-4-yl)-4-chloro-5-methylphenyl]proply}acetamide (1-7b)

To the single enantiomer of tert-butyl 4-(2-{1-[acetylamino]ethyl}-5-chloro-4-methylphenyl)piperidine-1-carboxylate 1-6b (220 mg, 0.522 mmol) was added 4 N HCl in dioxane (10 mL) at RT. The mixture was stirred at room temperature for 60 min, then the volatiles were removed under reduced pressure. Part of the residue (70 mg, 0.198 mmol) was dissolved in dichloromethane (5 mL) and (3S,4R)-1-tert-butyl-4-(2,4-difluorophenyl)pyrrolidine-3-carboxylic acid H-5 (66.2 mg, 0.198 mmol), DIEA (0.138 mL, 0.636 mmol), HOAT (27 mg, 0.198 mmol), and HATU (151 mg, 0.396 mmol) were added. The mixture was stirred at room temperature overnight, then concentrated to give a residue. The residue was purified by preparative TLC eluting with 10% MeOH in dichloromethane to give compound 1-7b. Compound 1-7b was isolated and converted the HCl salt by treatment with 1N HCl in ether. Calc MW. for C₃₂H₄₂ClF₂N₃O₂: 596; Found: 597 (M+H).

Following procedures similar to that described above for Example 1, the following compounds were prepared:

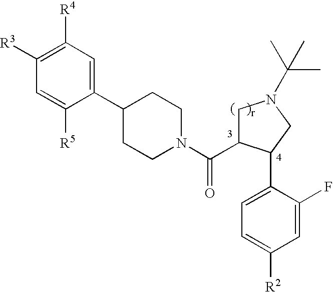

| | Stereo | | | | | | Parent ion |
|---|---|---|---|---|---|---|---|
| Ex # | (3,4) | r | R² | R³ | R⁴ | R⁵ | (M + H)⁺ |
| 2 | (S,R) | 1 | F | Me | F | 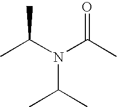 | 586 |
| 3 | (S,R) | 1 | F | Me | F | 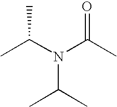 | 586 |
| 4 | (S,R) | 1 | Cl | Me | F | 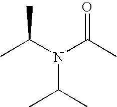 | 602 |
| 5 | (S,R) | 1 | F | Cl | Me | 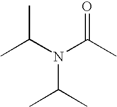 | 602 |
| 6 | (R,R) | 2 | F | F | Me | 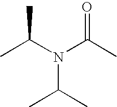 | 600 |
| 7 | (R,R) | 2 | F | F | Me | 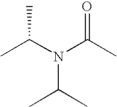 | 600 |
| 8 | (R,R) | 2 | F | Me | F | 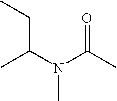 | 586 |
| 9 | (R,R) | 2 | F | Cl | Me | 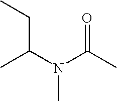 | 602 |

### EXAMPLE 10

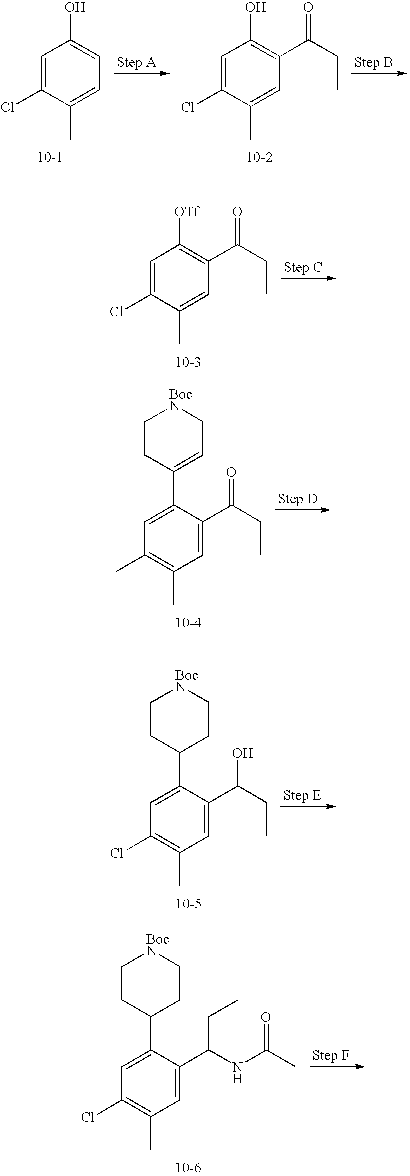

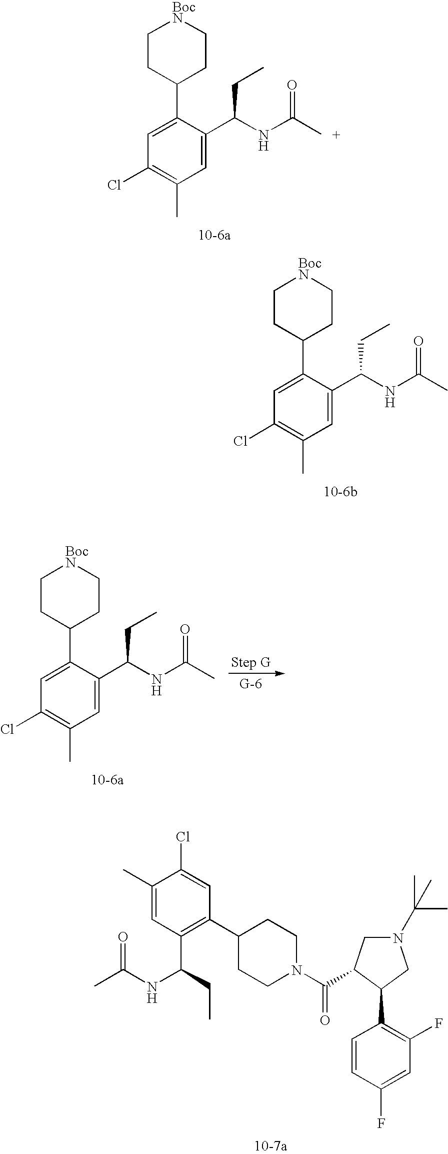

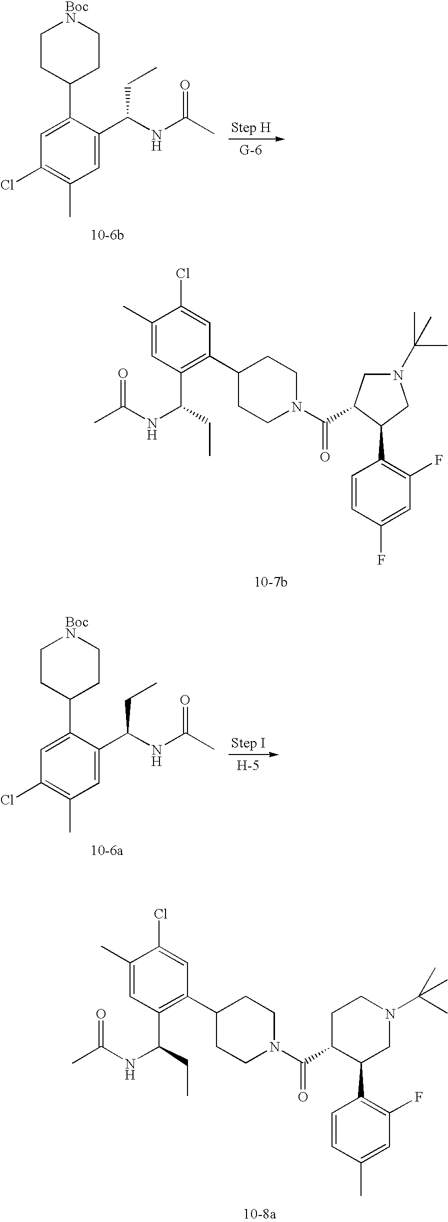

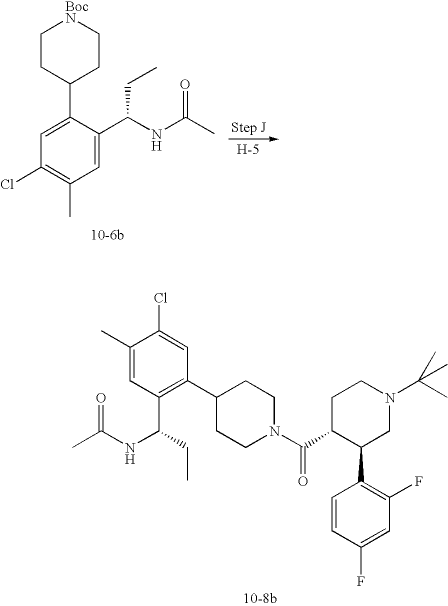

### Step A: Preparation of 1-(4-chloro-2-hydroxy-5 methylphenyl)propan-1-one (10-2)

To slurry of 3-chloro-4-methylphenol 10-1 (5.00 g, 35.1 mmol) and propionic chloride (3.35 mL, 38.6 mmol) was added aluminum trichloride (4.68 g, 35.1 mmol) portionwise and gas evolution began. When gas evolution ceased, the reaction was heated up to 180° C. for 1 h and the slurry became a yellow solid. The reaction mixture was cooled to room temperature and treated with a mixture of 25 mL of concentrated HCl aqueous solution and 100 mL of water. The suspension was stirred vigorously for 3 h and the fluffy solid was filtered and washed with cool water. The solid was then placed under high vacuum to dryness to give the titled compound 10-2.

### Step B: Preparation of 5-chloro-4-methyl-2-propionylphenyl trifluoromethanesulfonate (10-3)

To a solution of 1-(4-chloro-2-hydroxy-5-methylphenyl)propan-1-one 10-2 (5.50 g, 27.7 mmol) in methylene chloride (50 mL) was added dimethylamino-pyridine (0.338 g, 2.77 mmol) and the solution was cooled to −78° C. Triethyl amine (4.63 mL, 33.2 mmol) was added, followed by the dropwise addition of trifluoromethane sulfonic anhydride (5.45 mL, 9.14 mmol) over a period of 30 min, keeping the reaction temperature below −70° C. The reaction mixture was stirred at −78° C. for 30 min, then poured into ice-cooled water, and diluted with EtOAc. The resulting layers were separated; the aqueous layer was extracted with 2×200 mL EtOAc. The combined organic layers were washed with brine, dried over anhydrous MgSO₄, and concentrated to give a crude residue. Purification of the crude residue by flash chromatography (silica gel, 10% ethyl acetate/hexanes) afforded the titled compound 10-3 as a white wax-like solid.

### Step C: Preparation of tert-butyl 4-(5-chloro-4-methyl-2-propionylphenyl)-3,6-dihydropyridine-1(2H)-carboxylate (10-4)

To a mixture of 5-chloro-4-methyl-2-propionylphenyl trifluoromethanesulfonate 10-3 (9.00 g, 27.2 mmol), absolute ethanol (60 mL), toluene (60 mL), and 2M aqueous sodium carbonate (50 mL) was added tert-butyl 4-(4,4,5,5-tetramethyl-1,3,2-dioxaborolan-2-yl)-3,6-dihydropyridine-1(2H)-carboxylate (8.42 g, 27.2 mmol). The mixture was evacuated and flushed with nitrogen three times. After addition of tetrakis(triphenylphosphine)palladium(0) (3.16 g, 2.72 mmol), the mixture was heated at 75° C. for 1 h. After cooling to room temperature, the mixture was poured into water and extracted with EtOAc (3×250 mL). The combined organic layers were washed with brine, dried over anhydrous MgSO₄, filtered, and concentrated to give a black oil. The crude product was purified via flash column chromatography (silica gel, 10% EtOAc in hexane) to give the titled compound 10-4 as colorless oil.

### Step D: Preparation of tert-butyl 4-[5-chloro-2-(1-hydroxypropyl)-4-methylphenyl]piperidine-1-carboxylate (10-5)

A solution of tert-butyl 4-(3-chloro-4-methyl-2-propionylphenyl)-piperid-3-ene-1-carboxylate 10-4 (6.90 g, 19.0 mmol) in 200 mL of ethanol was added platinum oxide (0.431 mg, 1.90 mmol). After purging with hydrogen three times, the mixture was stirred overnight under hydrogen at atmospheric pressure at room temperature. The resulting solid was filtered and washed with EtOH three times. The filtrates were combined and concentrated to give crude product. The crude product was purified by a flash column chromatography (silica gel, 10% to 20% EtOAc/hexane gradient elution) to give the titled compound 10-5 as a white solid.

### Step E: Preparation of tert-butyl 4-{2-[1-(acetylamino)propyl]-5-chloro-4-methylphenyl]piperidine-1-carboxylate (10-6)

To a solution of tert-butyl 4-[5-chloro-4-methyl-2-(1-hydroxy-3-methylbutyl)phenyl]piperidine-1-carboxylate 10-5 (2.00 g, 5.44 mmol) in acetonitrile (60 mL) was added concentrated H₂SO₄ 81.5 mmol) in 30 mL of acetonitrile. The mixture was stirred at 60° C. overnight. After cooling to room temperature, the mixture was quenched with water and stirred for 30 min, followed by addition of aqueous 5N NaOH until the mixture pH was pH 9. The mixture was extracted with EtOAc (3×150 mL) and the combined organic layers were dried over Na₂SO₄, filtered, and concentrated to give a residue. The residue was dissolved in dichloromethane (20 mL), and Boc₂O (1.78 g, 8.15 mmol) and triethylamine (10.0 mL) were added. The mixture was stirred at room temperature overnight, then concentrated. Purification of the resulting crude residue by flash chromatography (silica gel, gradient elution: 5 to 20% isopropanol/hexanes) afforded the titled compound 10-6 as oil.

### Step F: Preparation of the single enantiomers of tert-butyl 4-{2-[1-(acetylamino)propyl]-5-chloro-4-methylphenyl]piperidine-1-carboxylate (10-6a and 10-6b)

The racemic mixture of tert-butyl 4-{2-[1-(acetylamino)propyl]-5-chloro-4-methylphenyl}piperidine-1-carboxylate 10-6 (2.00 g, 4.89 mmol) was separated using high performance liquid chromatography over ChiralCel AD 20×250 mm (˜50 mg per injunction; isocratic elution, 3% EtOH in heptane; flow rate=9 mL/min; UV detector wavelength=254) to afford the titled compounds 10-6a and 10-6b as white solids.

### Step G: Preparation of the single diastereomers of N-{1-[2-(1-{[(3S,4R)-1-tert-butyl-4-(2.4-difluorophenyl)pyrrolidin-3-yl]carbonyl}piperidin-4-yl)-4-chloro-5-methylphenyl]propyl}acetamide (10-7a)

To a solution of the single enantiomer of tert-butyl 4-(2-{1-[acetylamino}-ethyl}-5-chloro-4-methylphenyl)piperidine-1-carboxylate 10-6a (0.050 g, 0.122 mmol) in dichloromethane (0.1 mL) was added 4 N HCl in dioxane (1.0 mL). The mixture was stirred at room temperature for 30 min, then the volatiles were removed under reduced pressure. The resulting residue was dissolved in dichloromethane (5 mL) and (3S,4R)-1-tert-butyl-4-(2,4-difluorophenyl)pyrrolidine-3-carboxylic acid G-6 (0.042 g 0.147 mmol), HATU (0.056 g, 0.147 mmol), HOAT (0.020 g, 0.147 mmol), and DIEA (0.108 mL, 0.636 mmol) were added. The mixture was stirred at room temperature overnight and the solvents were removed under vacuum. The resulting residue was purified with reversed phase semi-preparative HPLC. The eluting solution was adjusted to basic by the addition of 1N NaOH aqueous solution, and extracted with EtOAc three times. The combined organic layers were dried with anhydrous NaSO₄, filtered, and concentrated to give the titled compound 10-7a as white solid. ESI-MS calc. for C₃₂H₄₂ClF₂N₃₀₂: 573; Found: 574 (M+H).

### Step H: Preparation of the single diastereomer of N-{1-[2-(1-{[(3S,4R)-1-tert-butyl-4-(2,4-difluorophenyl)pyrrolidin-3-yl]carbonyl}piperidin-4-yl)-4-chloro-5-methylphenyl]propyl}acetamide (10-7b)

To a solution of the single enantiomer of tert-butyl 4-(2-{1-[acetylamino]-ethyl}-5-chloro-4-methylphenyl)piperidine-1-carboxylate 10-6b (0.140 g, 0.342 mmol) in dichloromethane (0.20 mL) was added 4 N HCl in dioxane (2.0 mL). The mixture was stirred at room temperature for 30 min, then the volatiles were removed under reduced pressure. The resulting residue was dissolved in dichloromethane (10 mL), and (3S,4R)-1-tert-butyl-4-(2,4-difluorophenyl)pyrrolidine-3-carboxylic acid -6(0.116 g, 0.411 mmol), HATU (0.156 g, 0.411 mmol), HOAT (0.056 g, 0.411 mmol), and DIFA (0.301 mL, 1.78 mmol) were added. The mixture was stirred at room temperature overnight and the solvents were removed under vacuum. The resulting residue was purified with reversed phase semi-preparative HPLC. The eluting solution was adjusted to basic by addition of 1N NaOH aqueous solution and extracted with EtOAc three times. The combined organic layers were dried with anhydrous NaSO₄, filtered, and concentrated to give the titled compound 10-7b as white solid. ESI-MS calc. for C₃₂H₄₂ClF₂N₃₀₂: 573; Found: 574 (M+H).

### Step 1: Preparation of the single diastereomer of N-{1-[2-(1-{[(3R,4R)-1-tert-butyl-3-(2,4-difluorophenyl)piperidin4-yl]carbonyl}piperidin-4-yl)-4-chloro-5-methylphenyl]propyl}acetamide (10-8a)

To a solution of the single enantiomer of tert-butyl 4-{2-[1-(acetylamino)-propyl]-5-chloro-4-methylphenyl}piperidine-1-carboxylate 10-6a (0.050 g, 0.122 mmol) in dichloromethane (0.1 mL) was added 4 N HCl in dioxane (1.0 mL). The reaction mixture was stirred at room temperature for 30 min, then the volatiles were removed under reduced pressure. The residue was dissolved in dichloromethane (5 mL) and (3R,4R)-1-tert-butyl-4-carboxy-3-(2,4-difluorophenyl)piperidinium chloride H-5 (0.049 g, 0.147 mmol), HATU (0.056 g, 0.147 mmol), HOAT (0.020 g, 0.147 mmol), and DIEA (0.103 mL, 0.611 mmol) were added. The mixture was stirred at room temperature overnight and the solvents were removed under vacuum. The resulting residue was purified by HPLC to give the titled compound 10-8a as white solid. ESI-MS calc. for C₃₃H₄₄ClF₂N₃₀₂: 573; Found: 588 (M+H).

### Step J: Preparation of the single diastereomer of N-{1-[2-(1-{[(3R,4R)-1-tert-butyl-3-(2,4-difluorophenyl)piperidin-4-yl]carbonyl}piperidin-4-yl)-4-chloro-5-methylphenyl]propyl}acetamide (10-8b)

To a solution of the single enantiomer of tert-butyl 4-(2-{1-[acetylamino]-ethyl}-5-chloro-4-methylphenyl)piperidine-1-carboxylate 10-6b (0.080 g, 0.196 mmol) in dichloromethane (0.1 mL) was added 4 N HCl in dioxane (1.0 mL). The reaction mixture was stirred at room temperature for 30 min, then the volatiles were removed under reduced pressure. The resulting residue was dissolved in dichloromethane (5 mL) and (3R,4R)-1-tert-butyl-4-carboxy-3-(2,4-difluorophenyl)-piperidinium chloride H-5 (0.078 g, 0.235 mmol), HATU (0.089 g, 0.235 mmol), HOAT (0.032 g, 0.235 mmol), and DIEA (0.165 mL, 0.978 mmol) were added. The mixture was stirred at room temperature overnight and the solvents were removed under vacuum. Purification of the resulting crude residue by HPLC gave the titled compound 10-8b as a white solid. ESI-MS calc. for C₃₃H₄₄ClF₂N₃O₂: 573; Found: 588 (M+H).

Following procedures similar to that described above for Example 10, the following compounds were prepared:

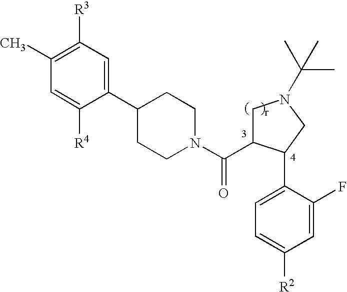

| | Stereo | | | | | Parent ion |
|---|---|---|---|---|---|---|
| Ex # | (3,4) | r | R² | R³ | R⁴ | (M + H)⁺ |
| 11 | (S,R) | 1 | F | F | 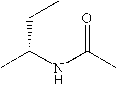 | 558 |
| 12 | (S,R) | 1 | F | F | 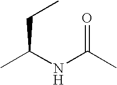 | 558 |
| 13 | (S,R) | 1 | Cl | F | 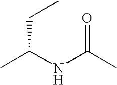 | 574 |
| 14 | (S,R) | 1 | Cl | Cl | 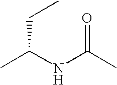 | 590 |
| 15 | (S,R) | 1 | Br | Cl | 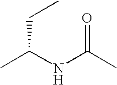 | 635 |
| 16 | (R,R) | 2 | Cl | Cl | 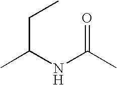 | 604 |

### EXAMPLE 17

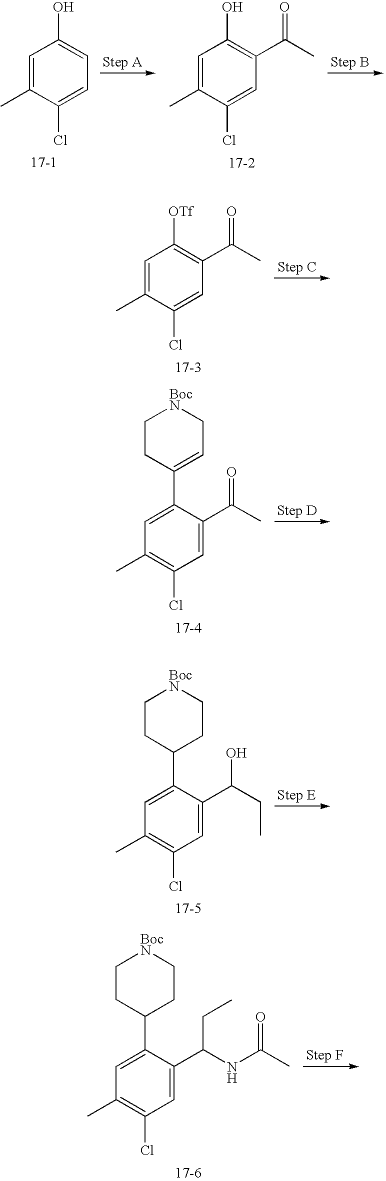

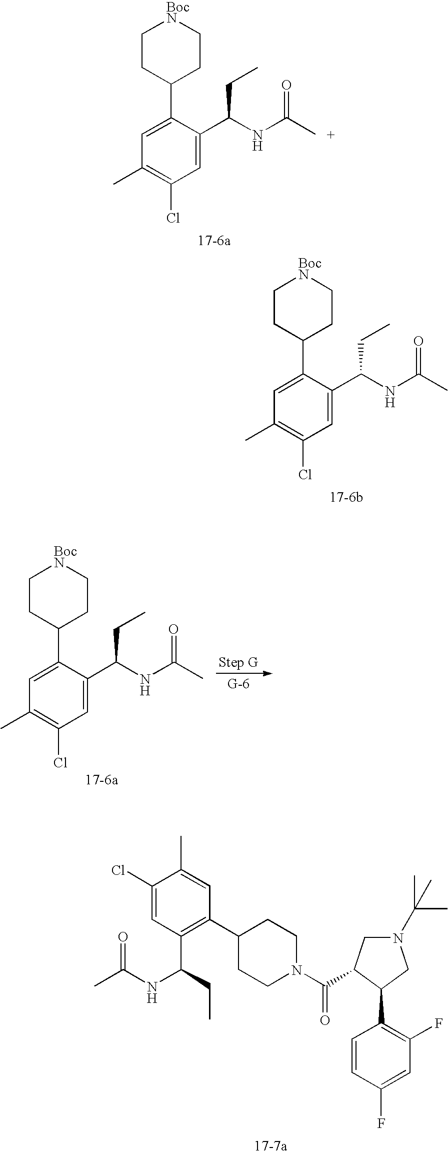

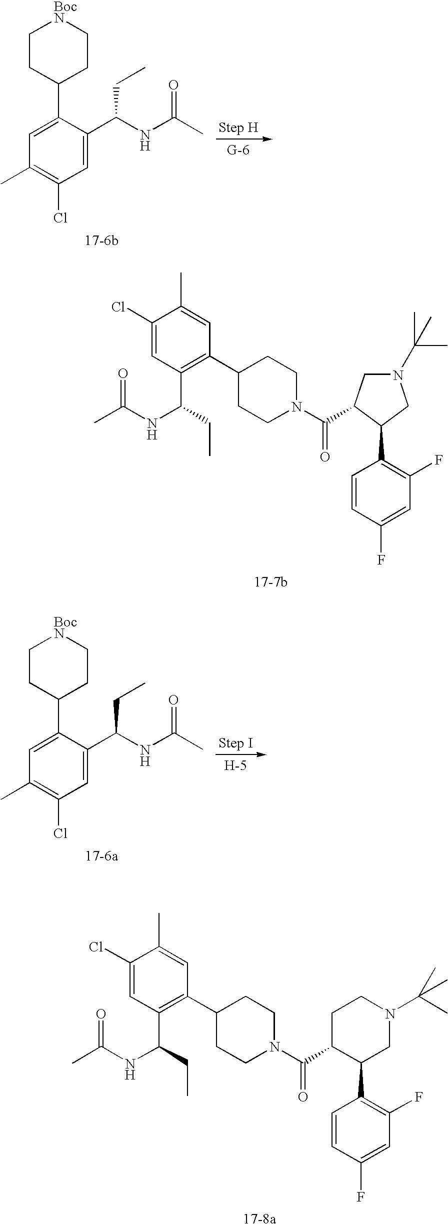

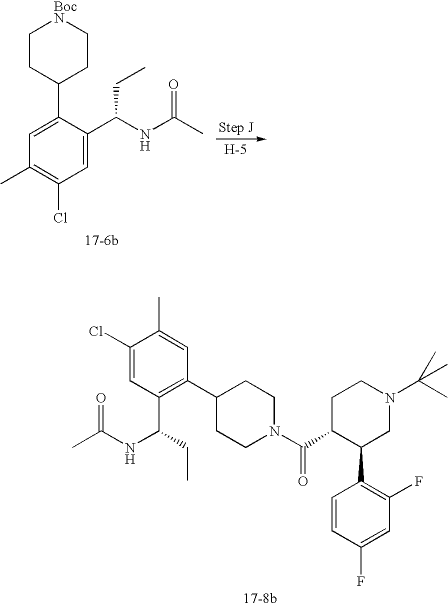

### Step A: Preparation of 1-(4-methyl-2-hydroxy-5-chlorophenyl)propan-1-one (17-2)

To slurry of 3-methyl-4-chlorophenol 17-1 (5.00 g, 35.1 mmol) and propionic chloride (3.35 mL, 38.6 mmol) was added aluminum trichloride (4.68 g, 35.1 mmol) portionwise and gas evolution began. When gas evolution ceased, the reaction was heated up to 180° C. for 1 hr and the reaction slurry became a yellow solid. The reaction mixture was cooled to room temperature and treated with a solution of 25 mL concentrated HCl/100 mL water. The suspension was stirred vigorously for 3 h, and the resulting fluffy solid was filtered and washed with cool water. The solid was dried under high vacuum overnight to give the titled compound 17-2 as an off-white solid.

### Step B: Preparation of 5-methyl-4-chloro-2-propionylphenyl trifluoromethanesulfonate (17-3)

To a solution of 1-(4-methyl-2-hydroxy-5-chlorophenyl)propan-1-one 17-2 (5.50 g, 27.7 mmol) in methylene chloride (50 mL) was added dimethylamino-pyridine (0.338 g, 2.77 mmol). The solution was cooled to −78° C., then triethyl amine (4.63 mL, 33.2 mmol) was added, followed by the dropwise addition of trifluoromethane sulfonic anhydride (5.45 mL, 9.14 mmol) over a period of 30 min, while keeping the reaction temperature below −70° C. The reaction was stirred at −78° C. for 30 min, then poured into ice-cooled water, and diluted with EtOAc. The layers were separated; the aqueous layer was extracted with 2×200 mL EtOAc. The combined organic layers were washed with brine, dried over anhydrous MgSO₄, and concentrated to give a crude residue. Purification of the crude residue by flash chromatography (silica gel, 10% ethyl acetate/hexanes) afforded the title compound 17-3 as a white solid.

### Step C: Preparation of tert-butyl 4-(5-methyl-4-chloro-2-propionylphenyl)-3,6-dihydropyridine-1(2H)-carboxylate (17-4)

To a mixture of 5-methyl-4-chloro-2-propionylphenyl trifluoromethanesulfonate 17-3 (9.00 g, 27.2 mmol), absolute ethanol (60 mL), toluene (60 mL), and 2M sodium carbonate (50 mL) was added tert-butyl 4-(4,4,5,5-tetramethyl-1,3,2-dioxaborolan-2-yl)-3,6-dihydropyridine-1(2T)-carboxylate (8.42 g, 27.2 mmol). The mixture was evacuated and flushed with nitrogen three times. After addition of tetrakis(triphenylphosphine)palladium(0) (3.16 g, 2.72 mmol), the mixture was heated at 75° C. for 1 h. After cooling to room temperature, the mixture was poured into water and extracted with EtOAc (3×250 mL). The combined organic layers were washed with brine, dried over anhydrous MgSO₄, filtered, and concentrated to give a black oil. The crude product was purified by flash column chromatography (silica gel, 10% EtOAc/hexane) to give the title compound 17-4 as a colorless oil.

### Step D: Preparation of tert-butyl 4-[5-methyl-2-(1-hydroxypropyl)-4-chlorophenyl]piperidine-1-carboxylate (17-5)

A solution of tert-butyl 4-(3-methyl-4-chloro-2-propionylphenyl)-piperid-3-ene-1-carboxylate 17-4 (6.90 g, 19.0 mmol) in 200 mL of ethanol was added platinum oxide (0.431 mg, 1.90 mmol). After purging with hydrogen three times, the mixture was stirred overnight under hydrogen at atmospheric pressure at room temperature. The solid was filtered off and washed with EtOH three times. The combined filtrates were concentrated to give a crude product. The crude product was purified by flash column chromatography (silica gel, gradient elution: 10% to 20% EtOAc/hexane) to give the titled compound 17-5 as a white solid.

### Step E: Preparation of tert-butyl 4-{2-[1-(acetylamino)propyl]-5-methyl-4-chlorophenyl}piperidine-1-carboxylate (17-6)

To a solution of tert-butyl 4-[5-methyl-4-chloro-2-(1-hydroxy-3-methylbutyl)phenyl]piperidine-1-carboxylate 17-5 (2.00 g, 5.44 mmol) in acetonitrile (60 mL) was added concentrated H₂SO₄ 81.5 mmol) in 30 mL of acetonitrile. The mixture was stirred at 60° C. overnight, then cooled to room temperature, and quenched with water. The mixture was stirred for 30 min, followed by addition of aqueous SN NaOH until the mixture pH was pH 9. The mixture was then extracted with EtOAc (3×150 mL), and the combined organic layers were dried over Na₂SO₄, filtered, and concentrated. The resulting residue was dissolved in dichloromethane (20 mL), and Boc₂O (1.78 g, 8.15 mmol) and triethylamine (10.0 mL) were added. The mixture was stirred at room temperature overnight, then concentrated to give a crude residue. Purification of the crude residue by flash chromatography (silica gel, gradient elution: 5 to 20% isopropanol/hexanes) afforded the titled compound 17-6 as white solid.

### Step F: Preparation of (1S)- and (1R)-tert-butyl 4-{2-[1-(acetylamino)propyl]-5-methyl-4-chlorolphenyl}piperidine-1-carboxylate (17-6a and 17-6b)

The racemic mixture of tert-butyl 4-{2-[1-(acetylamino)propyl]-5-methyl-4-chlorophenyl}piperidine-1-carboxylate 3-6 (2.00 g, 4.89 mmol) was separated using high performance liquid chromatography over ChiralCel AD 20×250 mm (˜50 mg per injunction; isocratic elution, 4% EtOH in heptane; flow rate=9 mL/min; UV detector wavelength=254) afforded the titled compounds 17-6a and 17-6b as white solids.

### Step G: Preparation of the single diastereomer of N-{1-[2-(1-{[(3S,4R)-1-tert-butyl-4-(2,4-difluorophenyl)pyrrolidin-3-yl]carbonyl}piperidin4-yl)-4-chloro-5-methylphenyl]propyl}acetamide (17-7a)

To a solution of the single enantiomer of tert-butyl 4-(2-{1-[acetylamino]-ethyl}-5-methyl-4-chlorophenyl)piperidine-1-carboxylate 17-6a (0.050 g, 0.122 mmol) in dichloromethane (0.1 mL) was added 4 N HCl in dioxane (1.0 mL) and the mixture was stirred at room temperature for 30 min and the volatiles were removed under reduced pressure to dryness. The residue was dissolved in dichloromethane (5.0 mL) and (3S,4R)-1-tert-butyl-4-(2,4-difluorophenyl)pyrrolidine-3-carboxylic acid G-6 (0.042 g, 0.147 mmol), HATU (0.056 g, 0.147 mmol), HOAT (0.020 g, 0.147 mmol), and DIEA (0.108 mL, 0.636 mmol) were added. The mixture was stirred at room temperature overnight and the solvents were removed under vacuum. The residue was purified with reversed phase semi-preparative HPLC. The eluting solution was adjusted to basic by addition of 1N NaOH aqueous solution and extracted with EtOAc three times. The combined organic layers were dried with anhydrous NaSO₄, filtered, and concentrated to give the title compound 17-7a as white solid. ESI-MS calc. for C₃₂H₄₂ClF₂N₃O₂: 573; Found: 574 (M+H).

### Step H: Preparation of the single diastereomer of N-{1-[2-(1-{[(3S,4R)-1-tert-butyl-4-(2,4-difluorophenyl)pyrrolidin-3-yl]carbonyl}piperidin-4-yl)-4-chloro-5-methylphenyl]propyl}acetamide (17-7b)

To a solution of the single enantiomer of tert-butyl 4-(2-{1-[acetylamino]-ethyl}-5-methyl-4-chlorophenyl)piperidine-1-carboxylate 17-6b (0.140 g, 0.342 mmol) in dichloromethane (0.20 mL) was added 4 N HCl in dioxane (2.0 mL). The mixture was stirred at room temperature for 30 min, then the volatiles were removed under reduced pressure. The residue was dissolved in dichloromethane (10 mL) and (3S,4R)-1-tert-butyl-4-(2,4-difluorophenyl)pyrrolidine-3-carboxylic acid G-6 (0.116 g, 0.411 mmol), HATU (0.156 g, 0.411 mmol), HOAT (0.056 g, 0.411 mmol), and DIEA (0.301 mL, 1.78 mmol) were added. The mixture was stirred at room temperature overnight and then concentrated in vacuo. The resulting residue was purified using reversed phase semi-preparative HPLC. The eluting solution was adjusted to basic by the addition of IN NaOH and extracted with EtOAc three times. The combined organic layers were dried with anhydrous NaSO₄, filtered, and concentrated to give the titled compound 17-7b as white solid. ESI-MS calc. for C₃₂H₄₂ClF₂N₃₀₂: 573; Found: 574 (M+H).

### Step I: Preparation of the single diastereomer of N-{1-[2-(1-{[(3R,4R)-1-tert-butyl-3-(2,4-difluorophenyl)piperidin-4-yl]carbonyl}piperidin-4-yl)-4-methyl-5-chlorophenyl]propyl}acetamide (17-8a)

To a solution of the single enantiomer of tert-butyl 4-{2-[1-(acetylamino)-propyl]-5-methyl-4-chlorophenyl}piperidine-1-carboxylate 17-6a (0.050 g, 0.122 mmol) in dichloromethane (0.1 mL) was added 4 N HCl in dioxane (1.0 mL). The mixture was stirred at room temperature for 30 min, then the volatiles were removed under reduced pressure. The resulting residue was dissolved in dichloromethane (5 mL), and (3R,4R)-1-tert-butyl-4-carboxy-3-(2,4-difluorophenyl)piperidinium chloride H-5 (0.049 g, 0.147 mmol), HATU (0.056 g, 0.147 mmol), HOAT (0.020 g, 0.147 mmol), and DIEA (0.103 mL, 0.611 mmol) were added. The mixture was stirred at room temperature overnight and then concentrated in vacuo to give a residue. The residue was purified by HPLC to give the titled compound 17-8a as white solid. ESI-MS calc. for C₃₃H₄₄ClF₂N₃₀₂: 587; Found: 588 (M+H).

### Step J: Preparation of the single diastereomer of N-{1-[2-(1-{[(3R,4R)-1-tert-butyl-3-(2,4-difluorophenyl)piperidin-4-yl]carbonyl}piperidin-4-yl)-4-methyl-5-chlorophenyl]propyl}acetamide (1 7-8b)

To a solution of the single enantiomer of tert-butyl 4-(2-{1-[acetylamino]-ethyl}-5-methyl-4-chlorophenyl)piperidine-1-carboxylate 17-6b (0.080 g, 0.196 mmol) in dichloromethane (0.1 mL) was added 4 N HCl in dioxane (1.0 mL). The mixture was stirred at room temperature for 30 min, then the volatiles were removed under reduced pressure. The resulting residue was dissolved in dichloromethane (5 mL), and (3R,4R)-1-tert-butyl-4-carboxy-3-(2,4-difluorophenyl)piperidinium chloride H-5 (0.078 g, 0.235 mmol), HATU (0.089 g, 0.235 mmol), HOAT (0.032 g, 0.235 mmol), and DIEA (0.165 mL, 0.978 mmol) were added. The mixture was stirred at room temperature overnight, then concentrated in vacuo to give a crude residue. Purification of crude residue by HPLC gave the titled compound 17-8b as a white solid. ESI-MS calc. for C₃₃H₄₄ClF₂N₃₀₂: 587; Found: 588 (M+H).

Following procedures similar to that described above for Example 17, the following compounds were prepared:

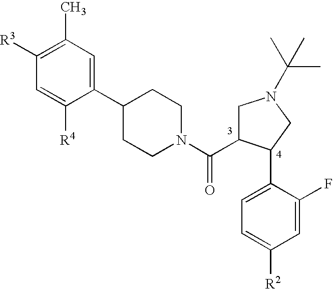

| | Stereo | | | | Parent ion |
|---|---|---|---|---|---|
| Ex # | (3,4) | R² | R³ | R⁴ | (M + H)⁺ |
| 18 | (S,R) | F | F | 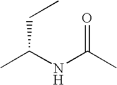 | 558 |
| 19 | (S,R) | Cl | F | 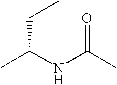 | 574 |
| 20 | (R,S) | Cl | F | 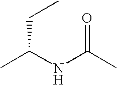 | 574 |

### EXAMPLE 21

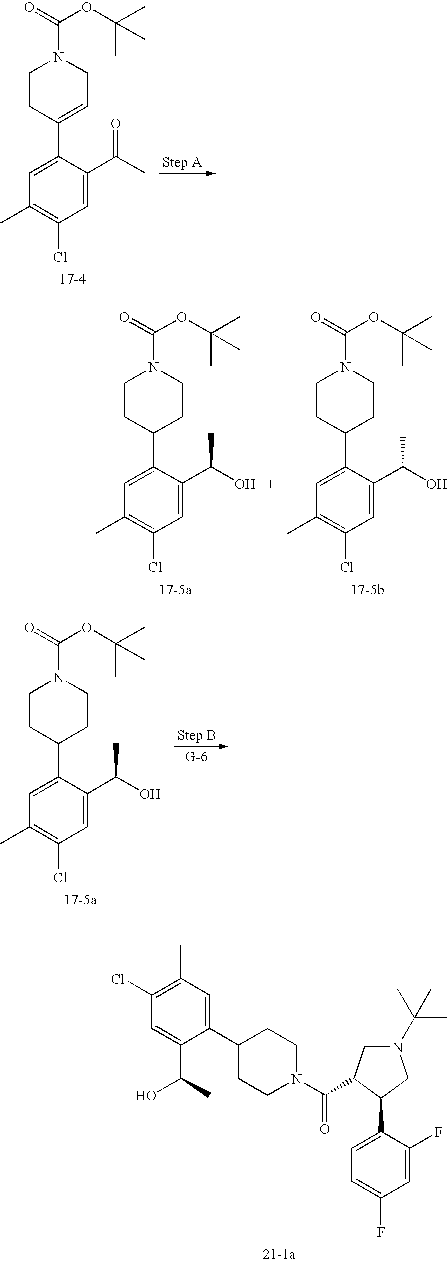

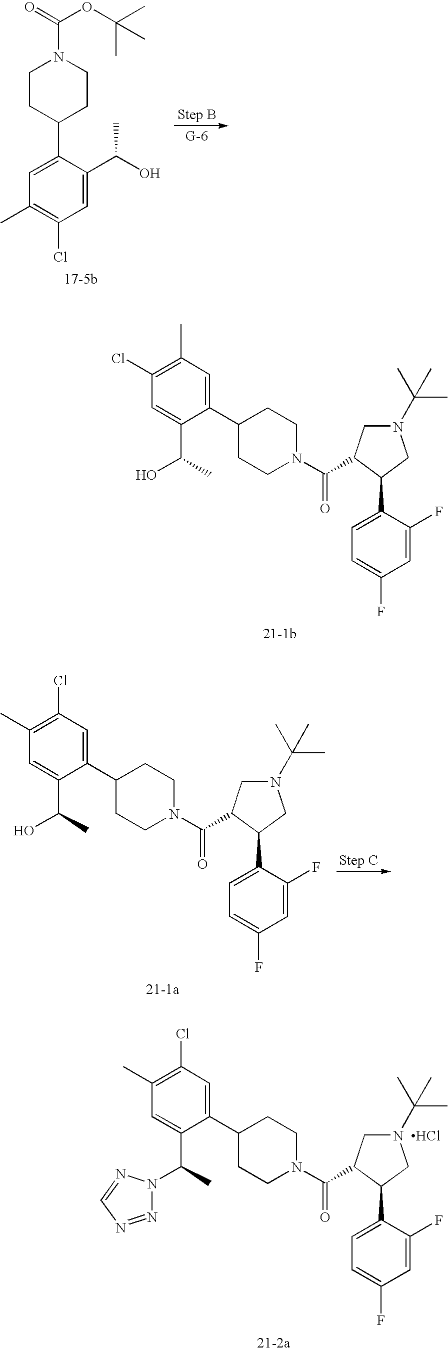

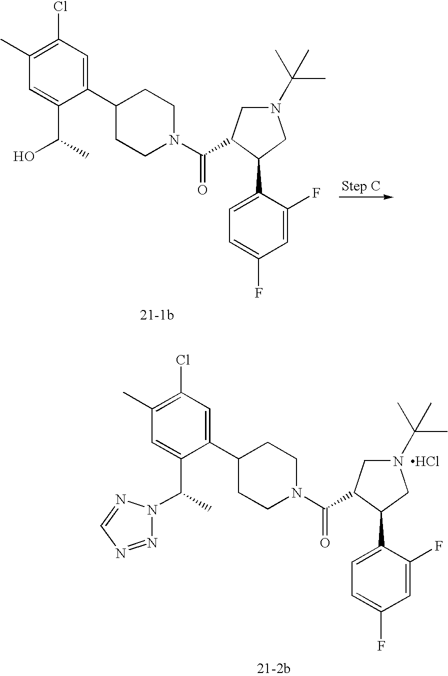

### Step A: Preparation of (17-5a) and (17-5b)

Platinum oxide (182 mg, 5 mol %) was added to a solution of compound 17-4 (5.6 g, 16.0 mmol) in ethanol (200 mL). After stirring under an atmosphere of hydrogen for 18 hr, the reaction was recharged with platinum oxide (182 mg, 5 mol %) and stirred for 24 hr. The reaction mixture was filtered through a short pad of Celite® and concentrated iii vacuo to give a crude residue. Purification of the crude residue by flash chromatography (silica gel, gradient elution: 2%-40% acetone/methylene chloride) afforded 17-5 as a mixture of diastereomers. The diastereomers were separated by BPLC (ChiralCel OJ 20>250 mm column eluting with 5% isopropanol/n-heptane at a flow rate at 9 mL per minute, 1 mL per injection of a 100 mg per mL solution) to afford the title compounds 17-5a and 17-5b as white solids (m/z (ES) 354 (MH⁺)).

### Step B: Preparation of (21-1a) and (21-1b)

Compound 21-1a was prepared from 17-5a following a similar procedure to that described for 17-7a (m/z (ES) 519 (MH⁺)).

Compound 21-1b was prepared from 17-5b following a similar procedure to that described for L7-7b (m/z (ES) 519 (MH⁺)).

### Step C: Preparation of (21-2a) and (21-2b)

Diisopropyl azodicarboxylate (0.21 mL, 1.08 mmol) was added dropwise over 30 min to a suspension of diphenylphosphino-polystyrene (2.2 mmol/g, 525 mg, 1.16 mmol) in a solution of tetrazole (3.43 mL, 0.45 M solution in acetonitrile, 1.54 mmol) and 21-1a (80 mg, 0.154 mmol) in methylene chloride (5 mL) at ambient temperature. After stirring at ambient temperature overnight, the reaction mixture was filtered and concentrated in vacuo to give a crude residue. Purification of the crude residue via flash chromatography (silica gel, gradient elution: 0%-20% methanol/methylene chloride) afforded 21-2a as a white solid. The solid was dissolved in the minimum amount of methylene chloride and acidified with hydrogen chloride (1M solution in diethyl ether), and concentrated in vacuo to afford the HCl salt of 21-2a as a white solid (m/z (ES) 571 (MH⁺)).

Compound 21-2b was prepared from 21-1b following a similar procedure to that described for 21-2a (m/z (ES) 571 (MH⁺)).

Following procedures similar to that described above for Example 21, the following compounds were prepared:

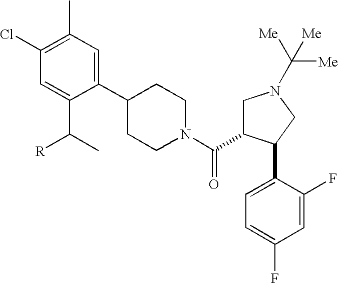

| | | Parent Ion m/z |
|---|---|---|
| Ex. # | R | (M + H) |
| 22 | 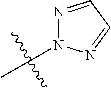 | 570 |
| 23 | 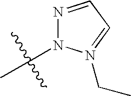 | 599 |

### Biological Assays

A. Binding Assay. The membrane binding assay was used to identify competitive inhibitors of ¹²⁵I-NDP-alpha-MSH ([Nle4, D-Phe7]-alpha-Melanocyte stimulating hormone) binding to cloned human MCRs expressed in mouse L- or Chinese hamster ovary (CHO)-cells.

Cell lines expressing melanocortin receptors were grown in T-180 flasks containing selective medium of the composition: 1 L Dulbecco's modified Eagles Medium (DMEM) with 4.5 g L-glucose, 25 mM Hepes, without sodium pyruvate, (Gibco/BR1); 100 ml 10% heat-inactivated fetal bovine serum (Sigma); 10 mL 10,000 unit/mL penicillin & 10,000 μg/mL streptomycin (Gibco/BR1); 10 ml 200 mM L-glutamine (Gibco/BR1); 1 mg/mL geneticin (G418) (Gibco/BR1). The cells were grown at 37° C. with CO₂ and humidity control until the desired cell density and cell number was obtained.

The medium was poured off and 10 mls/monolayer of enzyme-free dissociation media (Specialty Media Inc.) was added. The cells were incubated at 37° C. for 10 min or until cells sloughed off when flask was banged against hand.

The cells were harvested into 200 mL centrifuge tubes and spun at 1000 rpm, 4° C., for 10 min. The supernatant was discarded and the cells were resuspended in 5 mls/monolayer membrane preparation buffer having the composition: 10 mM Tris pH 7.2-7.4; 4 μg/mL Leupeptin (Sigma); 10 μM Phosphoramidon (Boehringer Mannheim); 40 μg/mL Bacitracin (Sigma); 5 μg/mL Aprotinin (Sigma); 10 mM Pefabloc (Boehringer Mannheim). The cells were homogenized with motor-driven dounce (Talboy setting 40), using 10 strokes and the homogenate centrifuged at 6,000 rpm, 4° C., for 15 min.

The pellets were resuspended in 0.2 mls/monolayer membrane prep buffer and aliquots were placed in tubes (500-1000 μL/tube) and quick frozen in liquid nitrogen and then stored at −80° C.

Test compounds or unlabelled NDP-α-MSH was added to 100 μL of membrane binding buffer to a final concentration of 1 μM. The membrane binding buffer had the composition: 50 mM Tris pH 7.2; 2 mM CaCl₂; 1 MM MgCl₂; 5 mM KCl; 0.2% BSA; 4 μg/mL Leupeptin (SIGMA); 10 μM Phosphoramidon (Boehringer Mannheim); 40 μg/mL Bacitracin (SIGMA); 5 μg/mL Aprotinin (SIGMA); and 10 mM Pefabloc (Boehringer Mannheim). One hundred μL of membrane binding buffer containing 10-40 μg membrane protein was added, followed by 100 μM 125I-NDP-α-MSH to final concentration of 100 μM. The resulting mixture was vortexed briefly and incubated for 90-120 min at room temp while shaking.

The mixture was filtered with Packard Microplate 196 filter apparatus using Packard Unifilter 96-well GF/C filter with 0.1% polyethyleneimine (Sigma). The filter was washed (5 times with a total of 10 mL per well) with room temperature of filter wash having the composition: 50 mM Tris-HCl pH 7.2 and 20 mM NaCl. The filter was dried, and the bottom sealed and 50 μL of Packard Microscint-20 was added to each well. The top was sealed and the radioactivity quantitated in a Packard Topcount Microplate Scintillation counter.

Representative compounds of the present invention were tested and found to bind to the melanocortin-4 receptor. These compounds were generally found to have IC₅₀ values less than 10 μM.

B. Functional assay. Functional cell based assays were developed to discriminate melanocortin receptor agonists from antagonists.

Cells (for example, CHO- or L-cells or other eukaryotic cells) expressing a human melanocortin receptor (see e.g. Yang-Y K; Ollmann-M M; Wilson-B D; Dickinson-C; Yamada-T; Barsh-G S; Gantz-I; Mol-Endocrinol. March 1997; 11(3): 274-80) were dissociated from tissue culture flasks by rinsing with Ca and Mg free phosphate buffered saline (14190-136, Life Technologies, Gaithersburg, Md.) and detached following 5 min incubation at 37° C. with enzyme free dissociation buffer (S-014-B, Specialty Media, Lavellette, N.J.). Cells were collected by centrifugation and resuspended in Earle's Balanced Salt Solution (14015-069, Life Technologies, Gaithersburg, Md.) with additions of 10 mM BEPES pH 7.5, 5 mM MgCl₂, 1 mM glutamine and 1 mg/ml bovine serum albumin. Cells were counted and diluted to 1 to 5×10⁶/mL. The phosphodiesterase inhibitor 3-isobutyl-1-methylxanthine was added to cells to 0.6 mM.

Test compounds were diluted in DMSO (10⁻⁵ to 10⁻¹⁰ M) and 0.1 volume of compound solution was added to 0.9 volumes of cell suspension; the final DMSO concentration was 1%. After room temperature incubation for 45 min, cells were lysed by incubation at 100° C. for 5 min to release accumulated cAMP.

cAMP was measured in an aliquot of the cell lysate with the Amersham (Arlington Heights, Ill.) cAMP detection assay (RPA556). The amount of cAMP production which resulted from an unknown compound was compared to that amount of cAMP produced in response to alpha-MSH which was defined as a 100% agonist. The EC₅₀ is defined as the compound concentration which results in half maximal stimulation, when compared to its own maximal level of stimulation.

Antagonist assay: Antagonist activity was defined as the ability of a compound to block cAMP production in response to alpha-MSH. Solution of test compounds and suspension of receptor containing cells were prepared and mixed as described above; the mixture was incubated for 15 min, and an EC₅₀ dose (approximately 10 nM alpha-MSH) was added to the cells. The assay was terminated at 45 min and cAMP quantitated as above. Percent inhibition was determined by comparing the amount of cAMP produced in the presence to that produced in the absence of test compound.

Representative compounds of the present invention were also tested in the functional assay and found generally to activate the melanocortin-4 receptor with EC₅₀ values less than 10 μM.

C. In vivo food intake and body weight models.

1) Food intake and body weight in rats. Sprague Dawley rats are administered test compound one hour prior to onset of dark cycle (12 hours). Food intake is determined either by measurement of the remaining amount of preweighed food the morning following the dosing or by using a computerized system in which each rat's food is placed on a computer monitored balance. Cumulative food intake for 16 h post compound administration is measured. In some cases, food intake measurements are followed as long as 2 weeks. Body weight is measured daily; in some cases, adiposity is measured by DEXAscan analysis, tissue weights and plasma drug levels are measured. Animals can be dosed by a number of routes of administration. The routes of administration include intravenous (IV), intraperitoneal (IP), subcutaneous (SC) and intracerebral ventricular (ICV).

Compounds useful in the present invention decrease food intake acutely by at least 20% and/or decrease body weight in a 2 week period by at least 4% relative to placebo.

2) Food intake in diet induced obese mice. Male C57/B16J mice maintained on a high fat diet (30-60% fat calories) are dosed with test compound for 1 to 30 days. Food intake and body weight are measured overnight and sometimes daily as long as 30 days. Biochemical parameters relating to obesity, including leptin, insulin, triglyceride, free fatty acid, cholesterol and serum glucose levels and pharmacokinetic parameters may be determined. Animals can be dosed by a number of routes of administration. The routes of administration include intravenous (IV), intraperitoneal (IP), subcutaneous (SC) and intracerebral ventricular (ICV).

Compounds useful in the present invention decrease body weight by at least 4% relative to placebo.

D. Male Sexual Dysfunction: Mouse electrically stimulated cavernosal nerve (ESCN) assay

Male C57BL6 mice are anesthetized, the carotid artery is exposed and cannulated for measurement of arterial pressure (MAP). A 30G needle attached to PE10 tubing, filled with heparinized saline, was inserted into the artery and glued in place. This tubing was connected to a pressure transducer and amplifier to measure direct MAP on a Gould 8 channel oscilloscope connected to a computer using the Po-ne-mah software to collect the data at one minute intervals. Another PE10 line attached to a 30G needle was inserted into the jugular vein for compound or vehicle administration. The cavernous nerve and penile body were exposed through a midline incision. Surrounding muscles were cauterized and removed for visualization of the cavernous nerve, which arises from the ipsilateral pelvic ganglion and is situated dorsal to the prostate. Another 30G needle attached to PE10 tubing, filled with heparinized saline, was inserted into the base of the corpus cavernosum near the crura and connected to the Gould system. A slight increase in intercavernous pressure (ICP) of approximately 5 to 10 mmHg is observed once this cannula is inserted into the corpus cavernosum. Heparinized saline (200 units/mL) was flushed through the cannula to assure proper placement of the cannula, inducing tumescence. The cavernous nerve was then isolated using curved #5 Dumont forceps and placed on a modified fixed position bipolar silver electrode (Harvard Apparatus). The electrodes are encased in plastic to allow stimulation of the nerve without additional stimulation of surrounding tissues. The electrode was advanced and held by a micromanipulator and was attached to a square wave stimulator to deliver electrical impulses at stimulation parameters ranging between 0.5 to 6.0 v, 2 to 16 Hz, 1 ms, for 30 seconds. Electrical stimulations were administered to individual animals with 5 minute intervals between stimulations. Responses reported at each time point represent the mean of the two stimulations. ICP, MAP and ICP/MAP responses were continuously recorded at one second intervals for the duration of the experiment.

Measurements of ICP, MAP and ICP/MAP ratio are analyzed and responses compared to nerve stimulation in the presence and absence of compound or vehicle. For each parameter monitored, responses evoked by duplicate electrical stimulations were averaged, and the mean values were used for comparison. Response segments of 10 s of baseline +30 s stimulation +150 s post-stimulation were used to evaluate changes in ICP in response to electrical stimulation of the cavernous nerve. To assess direct effects of compound administration on ICP, a 300 s pre-compound response segment was compared to a comparable segment immediately after compound administration.

Compounds useful in the present invention increase intracavernous pressure by at least 25% for a time period of at least 15 minutes relative to placebo.

E. Models of Female Sexual Dysfunction

Rodent assays relevant to female sexual receptivity include the behavioral model of lordosis and direct observations of copulatory activity. There is also a urethrogenital reflex model in anesthetized spinally transected rats for measuring orgasm in both male and female rats. These and other established animal models of female sexual dysfunction are described in McKenna K E et al, A Model For The Study of Sexual Function In Anesthetized Male And Female Rats, Am. J. Physiol. (Regulatory Integrative Comp. Physiol 30): R1276-R1285, 1991; McKenna K E et al, Modulation By Peripheral Serotonin of The Threshold For Sexual Reflexes In Female Rats, Pharm. Bioch. Behav., 40:151-156, 1991; and Takahashi L K et al, Dual Estradiol Action In The Diencephalon And The Regulation Of Sociosexual Behavior In Female Golden Hamsters. Brain Res., 359:194-207, 1985.

### EXAMPLE OF PHARMACEUTICAL COMPOSITIONS

As a specific embodiment of an oral composition of a composition of the present invention, 5 mg of Example 3 is formulated with sufficient finely divided lactose to provide a total amount of 580 to 1000 mg to fill a size O hard gelatin capsule.

As another specific embodiment of an oral composition of a compound of the present invention, 2.5 mg of Example 4 is formulated with sufficient finely divided lactose to provide a total amount of 580 to 1000 mg to fill a size O hard gelatin capsule.

While the invention has been described and illustrated in reference to certain preferred embodiments thereof, those skilled in the art will appreciate that various changes, modifications and substitutions can be made therein without departing from the spirit and scope of the invention. For example, effective dosages other than the preferred doses as set forth hereinabove may be applicable as a consequence of variations in the responsiveness of the mammal being treated for severity of bone disorders caused by resorption, or for other indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be limited only by the scope of the claims which follow and that such claims be interpreted as broadly as is reasonable.

1. A compound of structural formula I: 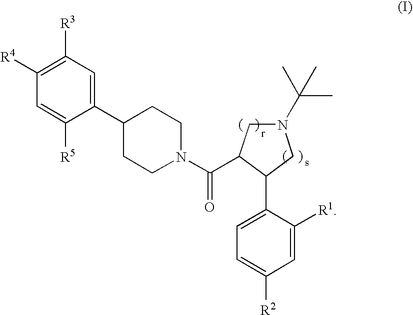
or a pharmaceutically acceptable salt thereof; wherein
R¹ and R² are selected from the group consisting of:
(1) halogen,
(2) CF₃,
(3) CH₃, and
(4) OCH₃;
R³ and R⁴ are independently selected from the group consisting of:
(1) —C₁₋₄ alkyl,
(2) —CF₃,
(3) halogen,
(4) —OC₁₋₄alkyl,
(5) —OCF₃,
(6) —OCHF₂,
(7) —S(O)ₚC₁₋₄alkyl, and
(8) —CN,
wherein alkyl is unsubstituted or substituted with one to three substituents independently selected from halogen, hydroxy, oxo, C₁₋₄ alkyl, trifluoromethyl, and C₁₋₄ alkoxy, or wherein the R³ and R⁴ substituents taken together with the carbons to which they are attached form a 4-6 membered ring optionally containing a heteroatom selected from O, S, —NH, and —NC₁₋₄alkyl;
R⁵ is selected from the group consisting of:
(1) —C₁₋₈ alkyl,
(2) —(CH₂)ₙ-heteroaryl,
(3) —(CH₂)ₙheterocycloalkyl,
(4) halogen,
(5) —OR⁶,
(6) —(CH₂)ₙC(O)R⁶,
(7) —(CH₂)ₙOC(O)R⁶,
(8) —(CH₂)ₙC(O)OR⁶,
(9) —(CH₂)ₙC≡N,
(10) —(CH₂)ₙN(R⁶)₂,
(11) —(CH₂)ₙC(O)N(R⁶)₂,
(12) —(CH₂)ₙNR⁶C(O)R⁶,
(13) —(CH₂)ₙNR⁶C(O)OR⁶,
(14) —(CH₂)ₙNR⁶C(O)-heteroaryl,
(15) —(CH₂)ₙNR⁶C(O)N(R⁶)₂,
(16) —(CH₂)ₙNR⁶-heteroaryl,
(17) —(CH₂)ₙC(O)NR⁶N(R⁶)₂,
(18) —(CH₂)ₙC(O)NR⁶NR⁶C(O)R⁶,
(19) —(CH₂)ₙNR⁶S(O)ₚR⁶,
(20) —(CH₂)ₙS(O)ₚN(R⁶)₂,
(21) —(CH₂)ₙS(O)ₚR⁶,
(22) —O(CH₂)ₙC(O)N(R⁶)₂,
(23) —(CH₂)ₙCF₃, and
(24) —O(CH₂)ₙCF₃,
wherein heteroaryl is unsubstituted or substituted with one to three substituents independently selected from halogen, hydroxy, C₁₋₄ alkyl, trifluoromethyl, and C₁₋₄ alkoxy, and wherein substituted with one to two substituents independently selected from halogen, hydroxy, oxo, C₁₋₄ alkyl, trifluoromethyl, and C₁₋₄ alkoxy, or two substituents on the same R⁵ carbon atom are taken together with the carbon atom to form a 3- to 6-membered ring;
each R⁶ is independently selected from the group consisting of:
(1) hydrogen,
(2) C₁₋₈ alkyl,
(3) phenyl,
(4) heteroaryl,
(5) —(CH₂)ₙheterocycloalkyl, and
(6) C₃₋₆ cycloalkyl,
wherein alkyl, phenyl, heteroaryl, heterocycloalkyl, and cycloalkyl are unsubstituted or substituted with one to three substituents independently selected from halogen, C₁₋₄ alkyl, hydroxy, and C₁₋₄ alkoxy, or two R⁶ substituents together with the atoms to which they are attached form a 4- to 8-membered mono- or bicyclic ring system optionally containing an additional heteroatom selected from O, S, —NH, and —NC₁₋₄ alkyl;
r is 1 or 2;
s is 0, 1, or 2;
n is 0, 1, 2, 3, or 4; and
p is 0, 1, or 2.

2. The compound according to claim 1 wherein R¹ is fluoro, and R² is selected from the group consisting of: fluoro and chloro; or a pharmaceutically acceptable salt thereof.

3. The compound according to claim 1 wherein R³ and R⁴ are independently selected from the group consisting of:
(1) methyl,
(2) fluoro, and
(3) chloro;
or a pharmaceutically acceptable salt thereof.

4. The compound according to claim 1 wherein R⁵ is selected from the group consisting of:
(1) —(CH₂)ₙ-heteroaryl, and
(2) —(CH₂)ₙNR⁶C(O)R⁶,
wherein heteroaryl is unsubstituted or substituted with one to three substituents independently selected from halogen, hydroxy, C₁₋₄ alkyl, trifluoromethyl, and C₁₋₄ alkoxy, and wherein any methylene (CH₂) carbon atom in R⁵ is unsubstituted or substituted with one to two substituents independently selected from halogen, hydroxy, oxo, C₁₋₄ alkyl, trifluoromethyl, and C₁₋₄ alkoxy, or two substituents on the same R⁵ carbon atom are taken together with the carbon atom to form a 3- to 6-membered ring.

5. The compound of claim 1 of structural formula IIa or IIb of the indicated trans relative stereochemical configuration: 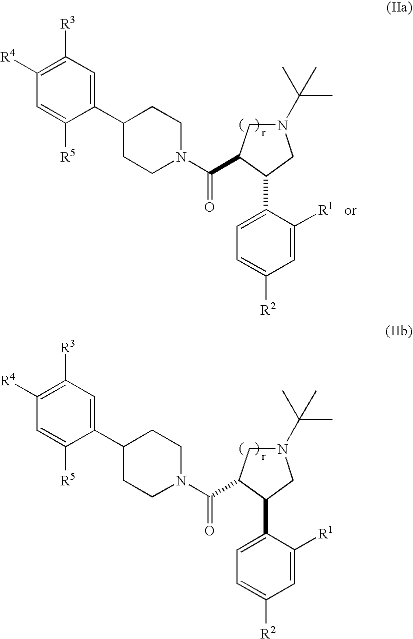
or a pharmaceutically acceptable salt thereof; wherein
R¹ and R² are selected from the group consisting of:
(1) chloro,
(2) fluoro,
(3) bromo,
(4) CF₃,
(5) CH₃, and
(6) OCH₃; and
R³, R⁴, R⁵, R⁶, r, n and p are as defined in claim 1.

6. The compound of claim 5 selected from the group consisting of: 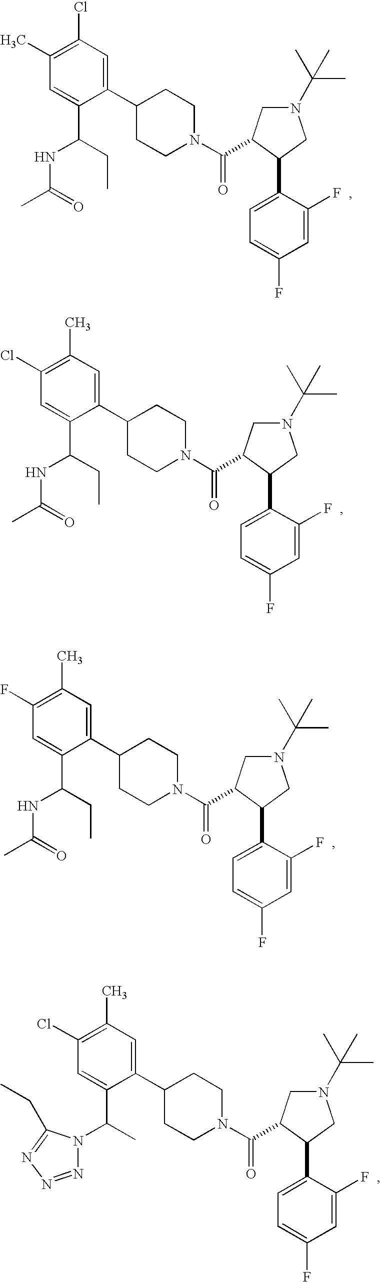 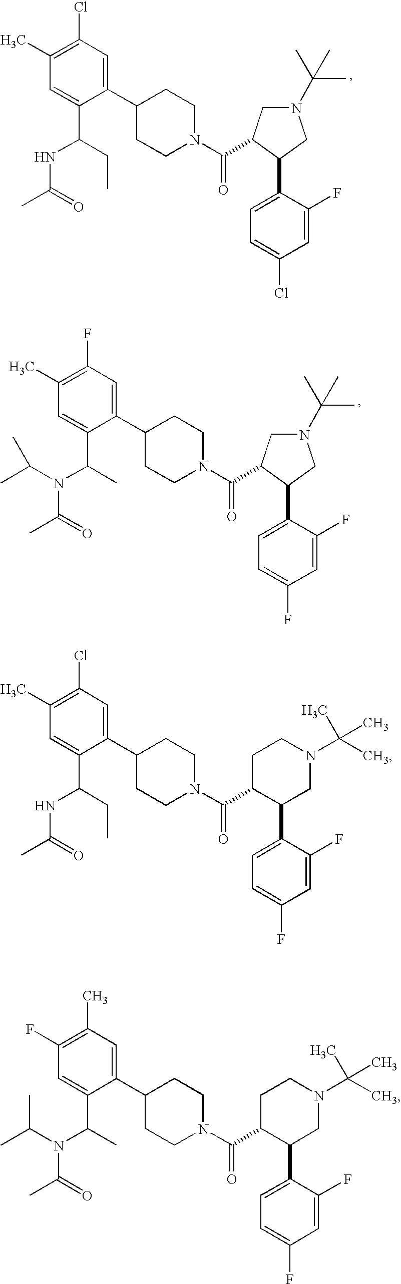
or a pharmaceutically acceptable salt thereof.

7. The compound of claim 6 which is: 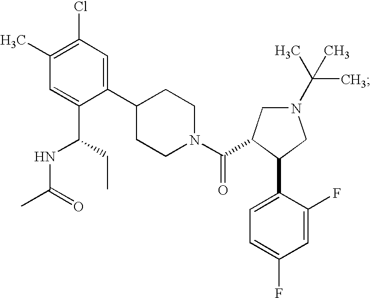
or a pharmaceutically acceptable salt thereof.

8. The compound of claim 6 which is: 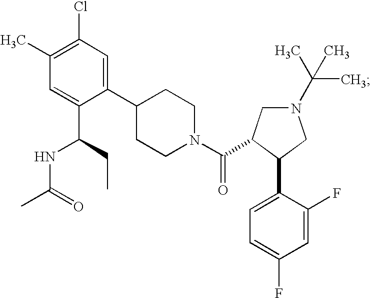
or a pharmaceutically acceptable salt thereof.

9. The compound of claim 6 which is: 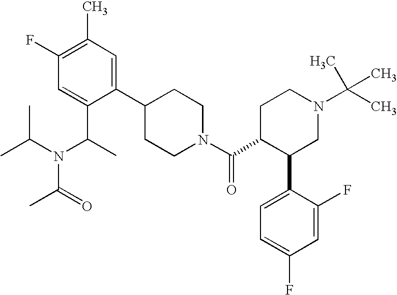
or a pharmaceutically acceptable salt thereof.

10. A pharmaceutical composition which comprises a compound of claim 1 and a pharmaceutically acceptable carrier.

11. The compound of claim 6 wherein the pharmaceutically acceptable salt is the hydrochloride salt.

12-15. (canceled)

16. A method for the treatment or prevention of obesity in a mammal in need thereof which comprises administering to said mammal a therapeutically or prophylactically effective amount of a compound of structural formula I.

17. A method of treating or preventing an obesity-related disorder in a mammal in need thereof which comprises administering to the mammal a therapeutically or prophylactically effective amount of a compound of structural formula I.

18. A method for the treatment or prevention of male or female sexual dysfunction in a mammal in need thereof comprising administering to the mammal a therapeutically or prophylactically effective amount of a compound of structural formula I.

19. A method for the treatment or prevention of erectile dysfunction in a mammal in need thereof comprising administering to the mammal a therapeutically or prophylactically effective amount of a compound of structural formula I.
